(19) United States
(12) Patent Application Publication
(10) Pub. No.: US 2014/0017810 A1
(43) Pub. Date: Jan. 16, 2014

(54) BIO-MOLECULE DETECTING DEVICE AND BIO-MOLECULE DETECTING METHOD

(71) Applicant: FUJIFILM Corporation, Tokyo (JP)

(72) Inventor: Toshihito KIMURA, Ashigarakami-gun (JP)

(21) Appl. No.: 13/934,325

(22) Filed: Jul. 3, 2013

Related U.S. Application Data
(63) Continuation of application No. PCT/JP2012/057192, filed on Mar. 21, 2012.

(30) Foreign Application Priority Data
Mar. 31, 2011 (JP) 2011-081247

(51) Int. Cl. G01N 33/543

(52) U.S. Cl. CPC G01N 33/54333; G01N 33/54373
USPC 436/501; 422/69

(57) ABSTRACT

A bio-molecule detecting device that enables a high-sensitivity measurement is provided. The orientation direction of third complexes included in blood plasma is switched by switching the vibration direction of an orientation control light. The orientation direction of the third complexes is switched between two directions in which the intensities of an electric field, which is generated between two gold nanoparticles included in the third complexes by surface plasmon resonance, are significantly different. Therefore, the intensity of fluorescence generated from the third complexes is significantly changed by the change in the orientation direction of the third complexes.

### BACKGROUND OF THE INVENTION

1. Field of the Invention

The present invention relates to a technique that detects detection subject substances in a solution, and particularly to a bio-molecule detecting device and a bio-molecule detecting method which can detect bio-molecules, viruses, DNAs, proteins, microbes and the like in a specimen, such as blood or urine.

2. Description of the Related Art

In recent years, attention has been paid to a bio-molecule detecting method that enables doctors, engineers and the like to detect bio-molecules in a medical examination place and immediately obtain measurement results, thereby helping diagnoses or cures. The bio-molecule detecting method is a method that selectively detects only detection subject substances in a body fluid including a plurality of components, such as blood, urine and sweat, using a high selectivity obtained by using a specific reaction, such as an antigen-antibody reaction. Particularly, the bio-molecule detecting method is widely used in the trace detection, inspection, quantity determination, analysis and the like of bio-molecules, such as viruses, DNAs, proteins and microbes.

In recent years, as a method for detecting bio-molecules at high sensitivity, studies are underway regarding a plasmon sensor in which an interaction between plasmons of fine metal particles and light is used.

JP2009-265062A discloses an analysis chip in which a phenomenon, in which absorption wavelengths of localized surface plasmon resonances of fine metal particles (gold nanorods) fixed to a substrate are shifted due to specific bonds, is applied to a sensing technique.

JP2007-248284A discloses a sensing element that improves a detecting sensitivity by orienting and fixing gold nanorods to a substrate.

### SUMMARY OF THE INVENTION

However, for the sensing element in which a fine metal particle-fixed substrate as described in JP2009-265062A and JP2007-248284A is used, there is a problem in that the cost of labor for fixing fine metal particles is high.

The invention has been made in consideration of the above described circumstance, and an object of the invention is to provide a bio-molecule detecting device and a bio-molecule detecting method which can perform a measurement at high sensitivity without fixing fine metal particles to a substrate.

In order to achieve the aforementioned object, a bio-molecule detecting device according to the invention includes a container that holds a solution including first complexes having a first substance that can specifically bond to a specific portion on a bio-molecule, metal particles and a fluorescent molecule and second complexes having a second substance that can specifically bond to a portion different from the specific portion on the bio-molecule, metal particles and a fluorescent molecule; an orientation control unit that orients third complexes, in which the first complexes bond to the bio-molecules through the first substances and the second complexes bond to the bio-molecules through the second substances in at least two directions in the solution; a light source that has a linear polarization component in a specific direction and radiates light, which causes surface plasmon resonance to the metal particles in the first complexes and the second complexes, on the solution; a light-receiving unit that detects fluorescence emitted from the fluorescent molecules in the first complexes and the second complexes by an electric field generated by the surface plasmon resonance of the metal particles in the first complexes and the second complexes; and a synchronous component-extracting unit that extracts a component synchronizing with an orientation cycle of the third complex in the fluorescence detected by the light-receiving unit.

When the bio-molecule detecting device has the above configuration, a detection subject substance can be measured at high sensitivity with a simple configuration. In addition, since the first complexes and the second complexes are not fixed, a reaction with bio-molecules in a solution is fast.

In addition, the orientation control unit preferably includes an orientation control polarized light source that radiates linearly polarized light, which is different from the light radiated from the light source, on the solution, and a polarizing axis-rotational moving unit that orients the third complexes in at least two directions in the solution by rotationally moving a polarizing axis of light radiated from the orientation control polarized light source.

When the orientation control unit orients the third complexes using light, it become unnecessary to carry out a pretreatment for orientation on the third complexes. For example, in a case in which the orientation is controlled using magnetism, it is necessary to bond magnetic particles and the like to the third complexes; however, when the third complexes are oriented using light, the above pretreatment becomes unnecessary. In addition, when the third complexes are oriented in a solution by rotationally moving the polarizing axis of light, it is not necessary to switch the orientation direction of the third complexes by radiating light from a plurality of directions, and therefore an optical system of the orientation control unit can be compacted.

In addition, an orientation control light source preferably radiates linearly polarized light, which is different from the light radiated from the light source, on the solution from a plurality of locations.

When the third complexes are oriented by radiating light on the solution from a plurality of locations, it becomes easy to control the orientation direction of the third complexes present at a variety of locations in the solution.

In addition, the orientation control unit may include an orientation control light source that radiates light, which is different from the light radiated from the light source, on the solution, and a switching unit that orients the third complexes in the solution in at least two directions by switching a radiation direction of light radiated from the orientation control light source. In addition, the orientation control light source preferably radiates light, which is different from the light radiated from the light source, on a plurality of locations of the solution.

In addition, the orientation control unit preferably orients the third complexes in a first direction, in which a long-axis direction of the third complexes and a vibration direction of the light radiated from the light source become parallel, and in a second direction, in which the long-axis direction of the third complexes and the vibration direction of the light radiated from the light source become vertical.

When the orientation of a molecule is controlled in the above manner, a change in a fluorescence intensity resulting from the switching of the orientation direction of the third complexes becomes largest. Therefore, the change in the fluorescence intensity resulting from the switching of the orientation direction of the third complexes enables high-sensitivity measurement.

In addition, the orientation control unit desirably changes the orientation direction of the third complexes at predetermined time intervals, and the synchronous component-extracting unit desirably extract a component synchronizing with the orientation cycle of the third complexes by measuring the intensity of fluorescence generated from the solution including the third complexes a plurality of times.

When the intensity of fluorescence is measured a plurality of times while changing the orientation direction of the third complexes at the predetermined time intervals as described above, and a plurality of the measured fluorescence intensities is arithmetically averaged or the like, the influence of the variation in the light extinction amount of each measurement, which is caused by noise or the like, on the measurement accuracy can be decreased.

Furthermore, the predetermined time interval is preferably a time interval during which the orientation of all third complexes present in the solution is completed.

When the predetermined time interval is determined in the above manner, measurement is not performed any longer when the orientation of all third complexes is completed, and therefore measurement can be carried out in the shortest period of time.

The wavelength of the light radiated from the light source is preferably a wavelength that is not absorbed by the fluorescent molecule. When the wavelength of the light radiated from the light source is a wavelength that is not absorbed by the fluorescent molecule, the fluorescent molecule is excited only by an electric field generated by surface plasmon resonance, and therefore the change of the intensity of the electric field is accurately reflected in the change of the intensity of fluorescence.

In addition, the synchronous component-extracting unit preferably extracts a component synchronizing with the orientation cycle of the third complexes using the fact that the amount of fluorescence generated from the third complexes changes by the change of the orientation direction of the third complexes.

In addition, the solution is preferably held in a container-holding unit having a flat plane at least in some part. Furthermore, the orientation control polarized light source preferably radiates linearly polarized light, which is different from the light radiated from the light source, in a direction in which the light outgoes from the flat plane of the container-holding unit through the solution, and focuses linearly polarized light, which is different from the light radiated from the light source, at an interface between the solution and the flat plane. In addition, the orientation control light source preferably radiates light, which is different from the light radiated from the light source, in a direction in which the light outgoes from the flat plane of the container-holding unit through the solution, and focuses light, which is different from the light radiated from the light source, at an interface between the solution and the flat plane.

When the orientation control polarized light source or the orientation control light source radiates light so as to focus the light at a location at which the light outgoes from the container-holding unit, the third complexes can be moved rotationally while pressing the third complexes on the wall surface of the container-holding unit, and therefore it becomes easy to control the orientation.

In addition, in order to achieve the above object, a bio-molecule detecting method according to the invention has a step of mixing a solution including first complexes having a first substance that can specifically bond to a specific portion on a bio-molecule included in a specimen, metal particles and a fluorescent molecule and second complexes having a second substance that can specifically bond to a portion different from the specific portion on the bio-molecule, metal particles and a fluorescent molecule and a specimen; a step of orienting third complexes, in which the first complexes bond to the bio-molecules through the first substances and the second complexes bond to the bio-molecules through the second substances in at least two directions in the solution; a step of radiating light, which has a linearly polarized component in a specific direction and causes surface plasmon resonance to the metal particles in the first complexes and the second complexes, on the solution; a step of detecting fluorescence emitted from the fluorescent molecules in the first complexes and the second complexes by an electric field generated by the surface plasmon resonance of the metal particles in the first complexes and the second complexes; and a step of extracting a component synchronizing with an orientation cycle of the third complexes in the fluorescence detected by the light-receiving unit.

According to the invention, since a solid phase is not used, an antigen-antibody reaction is fast, and an electric field generated by surface plasmon resonance is used in fluorescent detection, high-sensitivity bio-molecule detection can be carried out.

### BRIEF DESCRIPTION OF THE DRAWINGS

FIG. 1A is a schematic view of a first complex used in Embodiment 1, FIG. 1B is a schematic view of a second complex used in Embodiment 1, and FIG. 1C is a view illustrating a state in which the first complex and the second complex bond to an antigen.

FIGS. 2A and 2B is a schematic view illustrating the overview of an antigen-antibody reaction in a bio-molecule detecting device according to Embodiment 1.

FIG. 3A is a view expressing the intensity of an electric field generated around a gold nanoparticle when excitation light is radiated on the solely-present first complex. FIG. 3B is a view expressing the intensity of an electric field generated around a gold nanoparticle when an excitation light is radiated on a third complex having a long axis along an Y axis direction using gray images. FIG. 3C is a view expressing the intensity of an electric field generated around a gold nanoparticle when an excitation light is radiated on the third complex having a long axis along an X axis direction using gray images.

FIG. 4A is an outside perspective view of the bio-molecule detecting device according to Embodiment 1, and FIG. 4B is a view of the bio-molecule detecting device according to Embodiment 1 with an access cover open.

FIG. 5 is a function block diagram illustrating the principal configuration of the bio-molecule detecting device according to Embodiment 1.

FIG. 6A is a view illustrating the orientation direction of the third complexes in a case in which a polarization direction control unit allows orientation control light to pass through. FIG. 6B is a view illustrating the behavior of the third complexes in a case in which the vibration direction of the orientation control light is switched by 90 degrees. FIG. 6C is a view illustrating the orientation direction of the third complexes in a case in which the vibration direction of the orientation control light is switched by 90 degrees.

FIG. 7 is a view illustrating the location of the focus of the orientation control light.

FIG. 8A is a view illustrating the relationship between the orientation direction of the third complexes and the vibration direction of an excitation light in a case in which a polarization direction control unit allows orientation control light to pass through, and FIG. 8B is a view illustrating the relationship between the orientation direction of the third complexes and the vibration direction of an excitation light in a case in which the vibration direction of the orientation control light is switched by 90 degrees.

FIG. 9 is a graph drawing an orientation control signal outputted by a function generator (FG) during measurement, a light-receiving unit output outputted by a light-receiving unit, and a lock-in amplifier output outputted by a lock-in amplifier.

FIG. 10A is a schematic view illustrating another structure of the first complex, FIG. 10B is a schematic view illustrating another structure of the second complex, and FIG. 10C is a schematic view illustrating another structure of the third complex.

FIG. 11A is a schematic view illustrating the other structure of the first complex, FIG. 11B is a schematic view illustrating the other structure of the second complex, and FIG. 11C is a schematic view illustrating the other structure of the third complex.

FIG. 12A is a schematic view of a fourth complex used in Embodiment 2, FIG. 12B is a schematic view of a fifth complex used in Embodiment 2, and FIG. 12C is a view illustrating a state in which the fourth complex and the fifth complex bond to an antigen.

FIGS. 13A and 13B is a schematic view illustrating the overview of an antigen-antibody reaction in a bio-molecule detecting device according to Embodiment 2.

FIG. 14 is a block diagram illustrating the principal configuration of the bio-molecule detecting device according to Embodiment 2.

FIG. 15 is a schematic view expressing the detailed configuration of the light-receiving unit in the bio-molecule detecting device according to Embodiment 2.

FIG. 16A is a graph illustrating several cycles of an orientation control signal, several cycles of a light-receiving unit output and several cycles of a lock-in amplifier output in a case in which the third complexes are measured in the bio-molecule detecting device according to Embodiment 2. FIG. 16B is a graph illustrating several cycles of an orientation control signal, several cycles of a light-receiving unit output and several cycles of a lock-in amplifier output in a case in which sixth complexes are measured in the bio-molecule detecting device according to Embodiment 2.

FIG. 17 is a view illustrating a case in which orientation control light is radiated on multiple points in a reagent container.

FIG. 18 is a view illustrating the structure of an orientation control light source unit for entering orientation control light into multiple points.

FIG. 19 is a view illustrating an example of an optical system for entering orientation control light into multiple points.

FIG. 20 is a view illustrating another example of an optical system for entering orientation control light into multiple points.

FIG. 21 is a view illustrating a micro lens array.

### DESCRIPTION OF THE PREFERRED EMBODIMENTS

Hereinafter, embodiments of the invention will be described with reference to the accompanying drawings.

### Embodiment 1

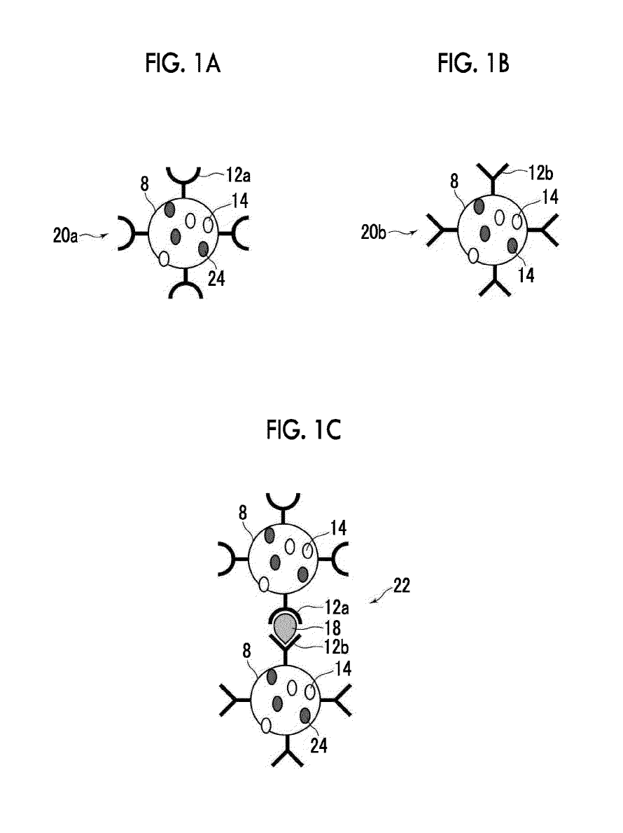

In Embodiment 1 of the invention, in order to detect an antigen present in a solution, two fluorescent labels 20a and 20b that can specifically bond to two specific places in the antigen are separately synthesized. FIG. 1A is a schematic view of the fluorescent label 20a, and FIG. 1B is a schematic view of the fluorescent label 20b. Since the fluorescent labels 20a and 20b are synthesized so as to include metal particles, when surface plasmon resonance occurs, fluorescence emanates. In the present embodiment, a specific antigen, which is a detection subject substance, is detected by introducing the two fluorescent labels into a homogeneous solution and bonding the labels to the antigen.

Hereinafter, the fluorescent label illustrated in FIG. 1A will be called a first complex 20a. The first complex 20a is a complex formed by covering the entire surface of a substantially spherical gold nanoparticle 8 having a diameter of 20 nm with a 10 nm-thick metal quenching prevention film made of SiO₂, and bonding a plurality of first antibodies 12a, a plurality of fluorescent molecules 14, and a plurality of bovine serum albumin (BSA) 24 to the surface of the metal quenching prevention film. The gold nanoparticle 8 is a core shell particle having Au as the core and a SiO₂ shell. The metal quenching prevention film is provided in order to prevent so-called metal quenching, in which the gold nanoparticle 8 and the fluorescent molecule 14 come close to each other and the gold nanoparticle 8 deprives the fluorescent molecule 14 of the excited energy. Meanwhile, the metal quenching prevention film is not particularly clearly illustrated in the drawing, and will not be particularly clearly illustrated in the following drawings either. The first antibodies 12a, the fluorescent molecules 14 and BSA 24 are evenly disposed on the entire surface of the metal quenching prevention film. BSA 24 is bonded in order to prevent non-specific adsorption, and prevents non-specific adsorption except the bond between the detection subject substance and the antibody 12a.

Hereinafter, the fluorescent label illustrated in FIG. 1B will be called a second complex 20b. The second complex 20b is a complex formed by covering the entire surface of the gold nanoparticle 8 having a diameter of 20 nm with a 10 nm-thick metal quenching prevention film made of SiO₂, and bonding a plurality of second antibodies 12b, a plurality of fluorescent molecules 14, and a plurality of BSA 24 to the surface of the metal quenching prevention film. The second antibodies 12b, the fluorescent molecules 14 and BSA 24 are evenly disposed on the entire surface of the metal quenching prevention film. As such, the first complex 20a and the second complex 20b employ a configuration with an isotropic shape.

The first antibody 12a and the second antibody 12b are antibodies that react with specific portions of an antigen, which is a detection subject substance, and bond to respectively different locations. In the embodiment, MAB 1129 (Human ErbB2/Her2 Antibody) manufactured by R&D Systems Inc. was used as the first antibody 12a, and BAF1129 (Human ErbB2/Her2 Biotinylated Antibody) manufactured by the same company was used as the second antibody 12b. In the embodiment, ErbB2/Her2 protein in blood plasma is detected as the detection subject substance. Hereinafter, ErbB2/Her2 protein, which is the detection subject substance, will be called the antigen 18.

Alexa Fluor 568 (product name of Molecular Probes) was used as the fluorescent molecule 14. Alexa Fluor 568 has a peak at a wavelength of approximately 600 nm, and emanates fluorescence having a wavelength of approximately 550 nm to 700 nm. The first complex 20a and the second complex 20b have the fluorescent molecule 14 of the same type.

A method for producing the first complex 20a will be described. First, the biotinylation of the first antibody 12a is carried out. Specifically, a One-Step Antibody Biotinylation Kit manufactured by Miltenyi Biotec is used. For example, a particle having Au as the core and a SiO₂ shell is manufactured using an Au colloid solution-SC with a particle diameter of 20 nm, manufactured by Tanaka Kikinzoku Kogyo, as the gold nanoparticle 8 and a micromixer method. A core shell particle having the entire surface of the gold nanoparticle 8 covered with a SiO₂ film can be produced in the above manner. An arbitrary well-known method can be used for the fixing of avidin to SiO₂. In addition, the first antibody 12a is fixed to the metal quenching prevention film through an avidin-biotin bond. Specifically, the particle and the antibody are mixed at the same ratio of the molar concentration, and idled for 1 hour. Subsequently, a fluorescent molecule is fixed to the metal quenching prevention film. The fluorescent molecule is fixed according to the protocol attached to a succeinimidyl ester reactive group kit manufactured by Molecular Probes. A9418-10G manufactured by SIGMA-ALDRICH is used as BSA 24. First, A9418-10G is adjusted to have a concentration of 100 times the molar concentration of the gold nanoparticle 8 using MillQ, A9418-10G and the gold nanoparticle 8 are mixed, and then idled for 1 hour, thereby completing the first complex 20a having the surface of the metal quenching prevention film blocked using BSA 24. The second complex 20b is also produced in the same order. In the present example, a method for fixing a fluorescent pigment to the surface of the gold nanoparticle having a SiO₂ shell is described, but there is another method in which the film thickness of SiO₂, which is a shell, is made to be as thick as approximately 20 nm, and a fluorescent pigment is included in the film. In addition, in a case in which the metal quenching prevention film is not provided, an avidin-labeled gold nanoparticle may be used.

The first antibody 12a and the second antibody 12b, which are used in the embodiment, are antibodies for detecting the antigen 18 by carrying out a so-called sandwich method. Therefore, the antibodies 18 are bonded to the antigen after labeled using respectively different epitopes. That is, the first antibody 12a and the second antibody 12b specifically bond to the respectively different locations of the antigen 18. In addition, the first antibody 12a and the second antibody 12b bond to far locations in the steric structure of the antigen 18 so as to prevent the antibodies from causing a steric hindrance to each other. Meanwhile, the first antibody 12a and the second antibody 12b in FIGS. 1A and 1B are illustrated using respectively different recess shapes, thereby indicating that the antibodies bond to different locations of the antigen 18. Therefore, the first antibody 12a and the second antibody 12b do not necessarily have such shapes.

FIG. 1C is a view illustrating a case in which both the first complex 20a and the second complex 20b bond to the antigen 18. Hereinafter, a complex, in which both the first complex 20a and the second complex 20b bond to the antigen 18 as illustrated in the drawing, will be called a third complex 22. In FIG. 1C, the antigen 18 is illustrated to have protrusion shapes matching the respective recess units of the first antibody 12a and the second antibody 12b in order to make easily understandable the fact that the first antibody 12a and the second antibody 12b specifically bond to respectively different locations of the antigen 18. The actual antigen 18 does not always have such shapes.

Since the first antibody 12a and the second antibody 12b are antibodies for carrying out the sandwich method on the antigen 18, in a case in which both the first complex 20a and the second complex 20b bond to the antigen 18, the first complex 20a and the second complex 20b bond to locations where the complexes face each other with the antigen 18 therebetween as illustrated in FIG. 1C. That is, the third complex 22 has a structure in which the first complex 20a, the antigen 18 and the second complex 20b are linearly arrayed. As such, the third complex 22 becomes a complex having an anisotropic shape. Hereinafter, in the third complex 22, a direction in which the first complex 20a, the antigen 18 and the second complex 20b are arrayed will be called a long-axis direction, and a vertical direction to the long-axis direction will be called a short-axis direction. In the third complex 22, the distance between the gold nanoparticle 8 included in the first complex 22a and the gold nanoparticle 8 included in the second complex 22b becomes approximately 30 nm.

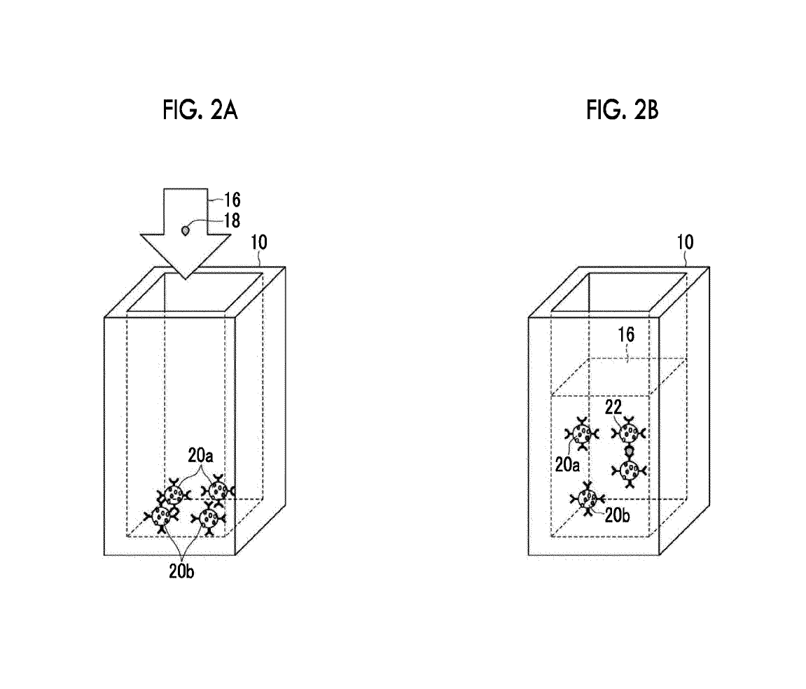

FIGS. 2A and 2B are schematic views illustrating the overview of an antigen-antibody reaction in a bio-molecule detecting device according to the embodiment. FIG. 2A illustrates a state before the antigen-antibody reaction. The reagent container 10 has a quadrangular prism-like outline, and has a reagent holding unit made up of a quadrangular prism-like recess portion with the opened top surface therein. In FIGS. 2A and 2B, the reagent holding unit, not exposed to the outside, is illustrated using broken lines. In the reagent holding unit, a plurality of dried first complexes 20a and a plurality of dried second complexes 20b are fed.

In the embodiment, the specimen is blood plasma 16 separated from whole blood. When the blood plasma 16 is injected into the reagent container 10, and stirred, in a case in which the antigen 18 that specifically bonds to the first antibody 12a and the second antibody 12b is present in the blood plasma 16, an antigen-antibody reaction is caused between the first antibody 12a and the second antibody 12b and the antigen 18, and the third complex 22 is formed as illustrated in FIG. 2B.

Since a sufficiently large amount of the first complexes 20a and the second complexes 20b are fed with respect to the antigen 18, some of the first complexes 20a and the second complexes 20b remain in the blood plasma 16 without causing an antigen-antibody reaction. That is, in the blood plasma 16, in which the first complexes 20a and the second complexes 20b are mixed, the first complexes 20a, the second complexes 20b and the third complexes 22 are present in a mixed state.

Meanwhile, components other than the antigen 18 are also present in the blood plasma 16, but components other than the antigen 18 will not be illustrated in FIGS. 2A and 2B in order to simplify the description.

A bio-molecule detecting device 100 according to the embodiment carries out the detection and quantity determination of the antigens 18 by radiating excitation light on the blood plasma 16, which is a liquid phase so that the first complexes 20a, the second complexes 20b and the third complexes 22 are present in a mixed state, causing surface plasmon resonance between the excitation light and the gold nanoparticle 8, emitting the light of the fluorescent molecule 14 using an electric filed generated by the surface plasmon resonance, and measuring fluorescence generated from the inside of the blood plasma 16.

Therefore, it is desirable to detect only the fluorescence generated from the third complexes 22 including the antigens 18, since the first complexes 20a, the second complexes 20b and the third complexes 22 are present in a mixed state in the blood plasma 16, when excitation light is radiated on the blood plasma 16, the fluorescent molecules 14 associated with the first complexes 20a and the second complexes 20b also generates fluorescence, which is an unnecessary component. Therefore, the bio-molecule detecting device 100 detects fluorescence while switching the orientation direction of the third complexes 22 using light, and computes the extent of contribution of fluorescence generated from the fluorescent molecules associated with the third complexes 22 in the entire fluorescence data based on the change in fluorescence intensity caused by the switching of orientation.

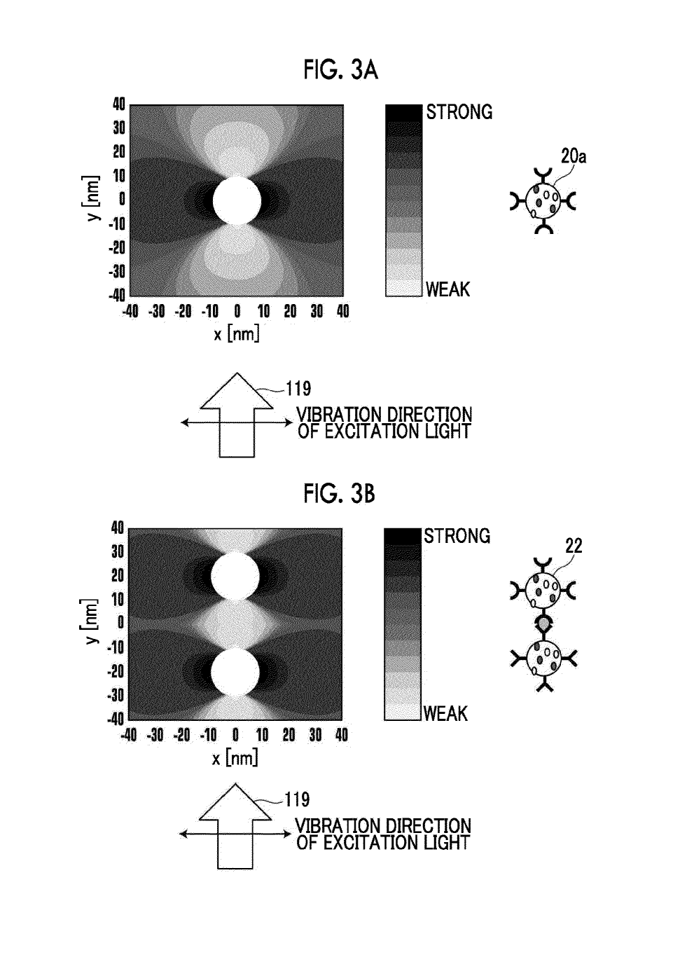

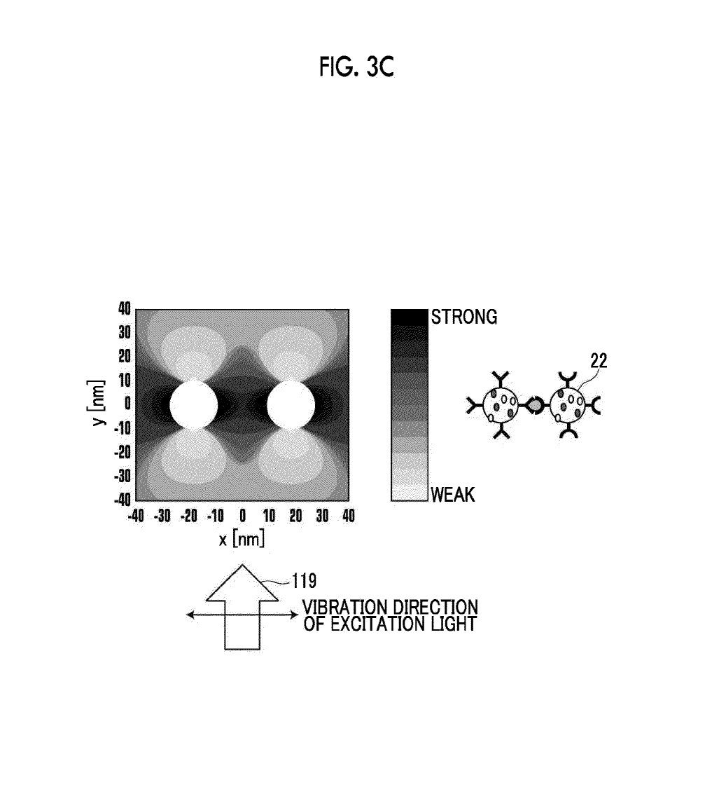

A principle for computing the extent of contribution of fluorescence generated from the fluorescent molecules 14 associated with the third complexes 22 and the extent of contribution of fluorescence generated from the fluorescent molecules 14 associated with the first complexes 20a and the second complexes 20b in the bio-molecule detecting device 100 will be described using FIGS. 3A to 3C. FIGS. 3A to 3C are views illustrating the intensities of electric fields generated by surface plasmon resonance of excitation light 119 and the gold nanoparticle 8 using gray images. The X axis and the Y axis indicate locations in FIGS. 3A to 3C. In addition, the density indicates the intensity of the electric field, and a dark density indicates that the electric field at the location is strong.

FIG. 3A is a view illustrating the intensity of the electric field generated around the gold nanoparticle 8 when the excitation light 119 travelling in the Y-axis direction is radiated on the solely-present first complex 20a while vibrating the complex in the X-axis direction using a gray image. The first complex 20a is present in the center of the gray image. In this case, an electric field is generated in the X-axis direction around the gold nanoparticle 8. That is, the electric field is generated in the same direction as the vibration direction of the excitation light 119 that generates surface plasmon resonance. The electric field is strongest in the vicinity of the surface of the gold nanoparticle 8, and becomes weak as the distance from the gold nanoparticle 8 increases. The fluorescent molecule 14 present on the surface of the gold nanoparticle 8 is excited using the generated electric field so as to generate fluorescence.

In the solely-present first complex 20a, since the shape is isotropic, and the fluorescent molecules 14 are evenly bonded to the entire surface of the metal quenching prevention film, the amount of generated fluorescence does not significantly changes even when the orientation changes. Meanwhile, the intensity distribution caused by the surface plasmon resonance of the second complex 20b is not illustrated, but becomes the same as the intensity distribution of the first complex 20a.

Meanwhile, in the third complex 22 having an anisotropic shape, the orientation can be controlled. FIG. 3B is a view illustrating the intensity of the electric field generated around the gold nanoparticle 8 when the excitation light 119 travelling in the Y-axis direction is radiated on the third complex 22 having the long-axis direction toward the Y-axis direction while vibrating the complex in the X-axis direction using a gray image. Even in this case, an electric field is generated in the X-axis direction around the gold nanoparticle 8. The intensities of the respective electric fields are almost the same as that when the first complex 20a or the second complex 20b is solely present. Therefore, the amount of fluorescence generated by the first complex 20a or the second complex 20b also becomes almost the same as that when the first complex 20a or the second complex 20b is solely present.

FIG. 3C is a view illustrating the intensity of the electric field generated around the gold nanoparticle 8 when the excitation light 119 travelling in the Y-axis direction is radiated on the third complex 22 having the long-axis direction toward the X-axis direction while vibrating the complex in the X-axis direction using a gray image. In this case, in an area between the gold nanoparticle 8 included in the first complex 20a and the gold nanoparticle 8 included in the second complex 20b, electric fields caused by surface plasmon resonance generated by the gold nanoparticles 8 included in the respective complexes overlap, and an extremely strong electric field is generated. The intensity of the electric field is approximately 20 times compared with the case of FIG. 3B. Therefore, the fluorescent molecules 14 present on the surface of the metal quenching prevention film and the area between the gold nanoparticle 8 included in the first complex 20a and the gold nanoparticle 8 included in the second complex 20b are excited by an extremely strong electric field, and strong fluorescence is generated. Therefore, the amount of fluorescence generated by the third complex 22 increases compared with the case of FIG. 3B. That is, in a case in which the excitation light 119 traveling in the Y-axis direction is radiated on the third complex while vibrating the complex in the X-axis direction, when the long-axis direction of the third complex 22 is changed from the Y-axis direction to the X-axis direction, the amount of fluorescence generated from the third complex 22 increases.

Therefore, the bio-molecule detecting device 100 according to the embodiment cyclically changes the orientation direction of third molecules using light, and detects only fluorescent signals synchronized with the above cycle, thereby computing the amount of fluorescence generated from the third complexes 22. Meanwhile, since the shapes of the first complex 20a and the second complex 20b are not anisotropic, the complexes are not orientated even when light is radiated. The first complexes 20a and the second complexes 20b rotate in the Brownian motion, but the amount of emitted fluorescence does not change even when the orientation changes, and therefore fluorescence synchronized with the cycle of the change of the orientation direction of the third molecules is not generated. Therefore, even in the blood plasma 16 in which the first complexes 20a, the second complexes 20b and the third complexes 22 are present in a mixed state, the extent of contribution of the fluorescence generated from the third complexes 22 can be computed.

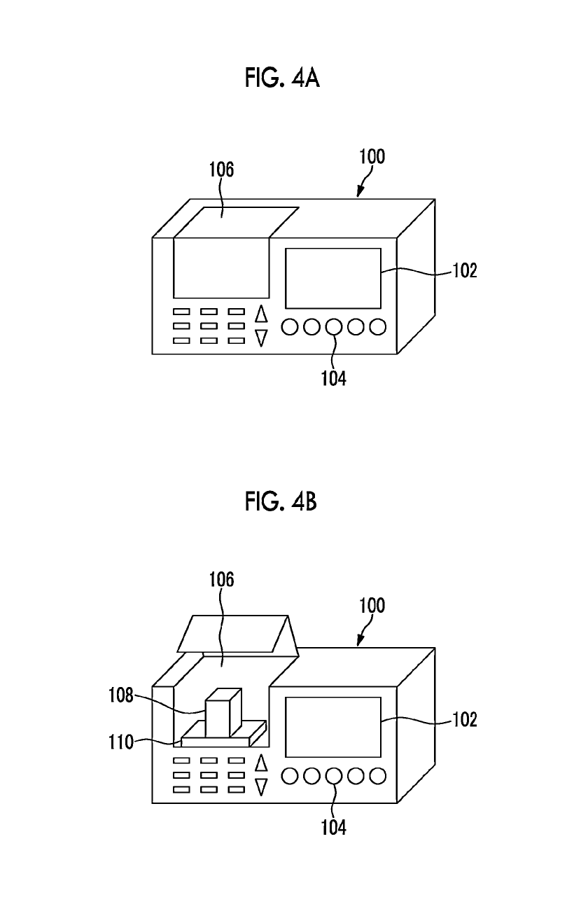

The configuration of the bio-molecule detecting device 100 that carries out the above treatment will be described. FIG. 4A is an outside perspective view of the bio-molecule detecting device 100. There are a display unit 102, a user input unit 104 and an access cover 106 on a side surface of the bio-molecule detecting device 100. The display unit 102 displays measurement results and the like. In the user input unit 104, setting of modes, input of specimen information, and the like are carried out. The access cover 106 is configured to be openable and closable, is opened when setting a specimen, and is closed during measurement. This configuration prevents external light from influencing measurement.

FIG. 4B is an outside perspective view of the bio-molecule detecting device 100 in a case in which the access cover 106 is opened. When the access cover 106 is opened, there are the reagent container 10 and a holding table 110 in the device. The reagent container 10 is held by the holding table 110, and is attachable to and detachable from the holding table 110. The reagent container 10 is a quadrangular prism-like container to which a solution is fed. A user injects a specimen into the reagent container 10, closes a lid, and carries out measurement. Although not illustrated, there are a reagent tank and a dispensing unit in the bio-molecule detecting device 100, and, when a measurement begins, the dispensing unit sucks up a reagent from the reagent tank, and dispenses the reagent into the reagent container 10.

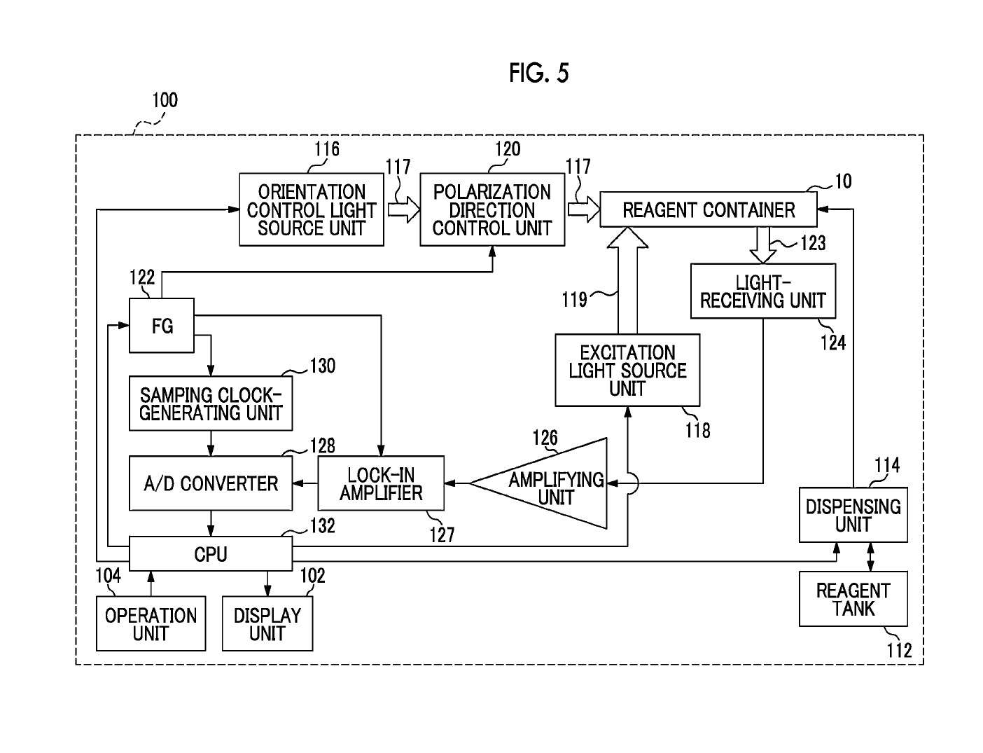

FIG. 5 is a function block diagram for explaining the principal configuration of the bio-molecule detecting device 100. The bio-molecule detecting device 100 has the display unit 102, the user input unit 104, the reagent container 10, a reagent tank 112, the dispensing unit 114, an orientation control light source unit 116, an excitation light source unit 118, a polarization direction control unit 120, a function generator (FG) 122, a light-receiving unit 124, an amplifying unit 126, a lock-in amplifier 127, an A/D converter 128, a sampling clock-generating unit 130 and a CPU 132.

The reagent container 10 is a container in which the reagent stored in the reagent tank 112 and a specimen sampled from a patient or the like are reacted. The reagent container 10 is attachable to and detachable from the bio-molecule detecting device 100. The capacity of the reagent container 10 is approximately 120 μL.

The reagent tank 112 is a tank that stores a plurality of kinds of reagents. The first complexes 20a and the second complexes 20b are stored in the reagent tank 112 as reagents.

The dispensing unit 114 is configured of a detachable pipette or aspirator. The dispensing unit 114 obeys orders from the CPU 132, sucks a reagent to be used in measurement from the reagent tank 112 using the pipette, and dispenses into the reagent container 10.

The orientation control light source unit 116 is a light source that radiates orientation control light 117, which is linearly polarized by internal polarizers, toward the polarization direction control unit 120. Here, the linearly-polarized light refers to light having a constant vibration direction and a fixed polarization plane. A surface on which the travelling direction and vibration direction of the linearly-polarized light are present is called the polarization plane, and the vibration direction of the linearly-polarized light is called a polarization axis. The orientation control light source unit 116 applies an external force to the third complexes 22 present in the solution in the reagent container 10 using the orientation control light 117, thereby orientating the third complexes 22. As the orientation control light 117, for example, a laser having a wavelength of 909 nm and an output of 700 mW is used. The orientation control light 117 is a laser having a wavelength, which is not absorbed by the fluorescent molecule 14, and has no influence, such as breaking the pigment of the fluorescent molecule 14. The orientation control light 117 has a width large enough to radiate the entire solution in the reagent container 10.

The external force generated by the orientation control light 117 is a force generated as a counteraction when the orientation control light 117 is hit and scattered on the third complexes 22. The third complex 22 forms a complex having an anisotropic shape as illustrated in FIG. 1C. Therefore, in a case in which the orientation control light 117 is hit on the third complex 22, the third complex 22 moves rotationally so that the counteraction with respect to the orientation control light 117 becomes smaller. As a result, the long-axis direction of the third complex 22 and the vibration direction of the orientation control light 117 become parallel. Since the third complex reaches the most energetically stable state when the above parallel relationship is formed, the rotational moving of the third complex 22 stops at this time. In other words, the third complexes 22 are dispersed in random directions in a solution when the orientation control light 117 is not radiated; however, when the orientation control light 117 is radiated, the third complex moves rotationally, and stops the rotational moving at a location at which the long-axis direction becomes parallel to the vibration direction of the orientation control light 117. Meanwhile, since the first complex 20a and the second complex 20b do not have an anisotropic shape, the complexes are not oriented.

The polarization direction control unit 120 switches the orientation direction of the third complexes 22 by switching the vibration direction of the orientation control light 117. The polarization direction control unit 120 has a half-wavelength plate. The half-wavelength plate is a phase plate having a function of changing the optical path difference of polarized light vibrating in the vertical direction by half wavelength, and is used to rotate the polarization plane of light. Light linearly polarized in a parallel direction to the optical axis direction of the half-wavelength plate passes through the half-wavelength plate, however, for light linearly polarized in a direction forming 45 degrees with the optical axis direction of the half-wavelength plate, the vibration direction changes by 90 degrees. That is, a case in which light is allowed to pass through and a case in which the vibration direction of light is changed by 90 degrees can be switched by switching the angle of the half-wavelength plate with respect to linearly polarized light. The polarization direction control unit 120 receives a signal from FG 122, rotationally moves the half-wavelength plate, and switches the vibration direction of the orientation control light 117 by 90 degrees. In other words, the vibration direction of the polarization control light 117 is determined by a voltage signal generated by FG 122. The polarization direction control unit 120 allows the orientation control light 117 to pass through in a case in which FG 122 outputs a signal of 0 V, and switches the vibration direction of the orientation control light 117 by 90 degrees in a case in which FG 122 outputs a signal of 5 V. Hereinafter, signals outputted to the orientation control unit 120 from FG 122 are called orientation control signals.

The excitation light source unit 118 is a light source that radiates the excitation light 119, which is linearly polarized by polarizers included in the excitation light source unit, upward from the bottom surface of the reagent container 10. The excitation light source unit 118 radiates the excitation light 119 so as to generate surface plasmon resonance between the excitation light 119 and the gold nanoparticle 8. As the excitation light 119, light having a wavelength of 635 nm and an output of 10 mW is used.

FG 122 is a device that can generate voltage signals having a variety of frequencies and waveforms. FG 122 receives orders outputted from CPU 132, and outputs voltage signals to the polarization direction control unit 120, the lock-in amplifier 127 and the sampling clock-generating unit 130.

CPU 132 controls a timing, at which the polarization direction control unit 120 switches the travelling direction of the orientation control light 117, by designating an orientation control signal outputted with respect to FG 122.

The light-receiving unit 124 is configured of a filter, a photodiode or the like. The light-receiving unit 124 is provided below the reagent container 10, receives fluorescence 123 generated from the fluorescent molecule 14 in the reagent container 10 below the reagent container 10, converts into an analog electric signal, and outputs to the amplifying unit 126. The filter in the light-receiving unit 124 is a filter that cuts light having a wavelength other than fluorescence generated from the fluorescent molecule 14.

The amplifying unit 126 amplifies analog fluorescent data outputted from the light-receiving unit 124, and outputs to the lock-in amplifier 127.

The lock-in amplifier 127 converts the frequency of the analog fluorescent data into a direct current. A square wave, which is a reference signal, is inputted to the lock-in amplifier 127 from FG 122. The lock-in amplifier 127 carries out detection of a frequency component, which is equal to the reference signal, from the analog fluorescent data outputted from the amplifying unit 126. Specifically, the lock-in amplifier 127 converts only the frequency component that is equal to the reference signal into a direct-current signal using synchronous detection, and allows only the direct-current signal to pass through using a lowpass filter provided therein. The lock-in amplifier 127 outputs the direct-current signal to the A/D converter 128.

The sampling clock-generating unit 130 inputs to the A/D converter 128 a sampling clock that designates a timing, at which the A/D converter 128 samples the analog fluorescent data, based on the voltage signal inputted from FG 122.

The A/D converter 128 carries out sampling of the analog fluorescent data outputted from the lock-in amplifier 127 based on the sampling clock outputted from the sampling clock-generating unit 130, converts a sample into digital data, and outputs to CPU 132.

CPU 132 carries out the computation of digital data outputted from the A/D converter 128, and outputs a result to the display unit 102. In addition, CPU 132 receives an input from the user input potion 104, instructs and orders the operation of the orientation control light source unit 116, the excitation light source unit 118, the dispensing unit 114 and FG 122. Specifically, CPU carries out the ordering of the ON and OFF of the orientation control light source unit 116 and the excitation light source unit 118, the ordering of designating a reagent to use and starting a dispensing operation with respect to the dispensing unit 114, and the ordering of instructing and outputting a signal waveform to output with respect to FG 122.

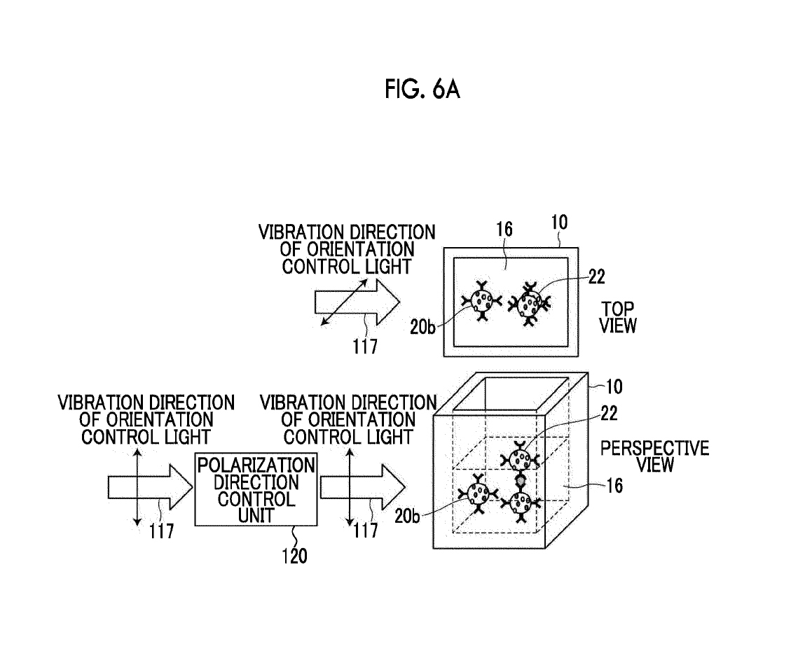

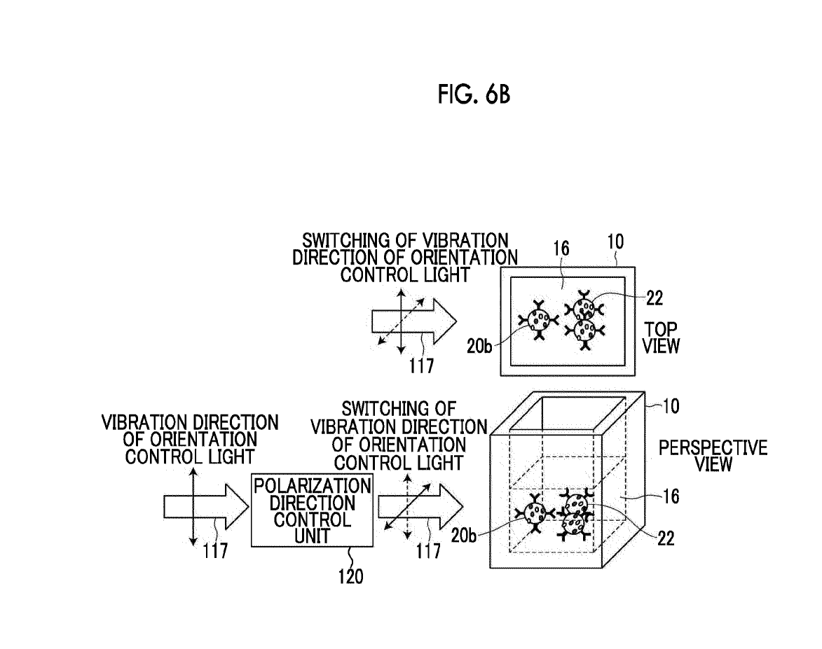

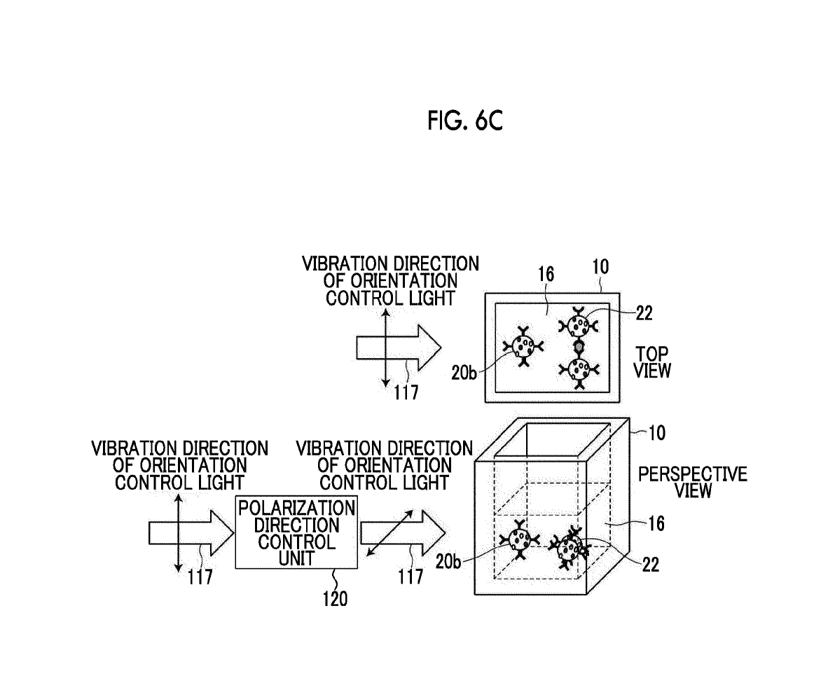

FIGS. 6A to 6C are views illustrating the orientation directions of the third complex 22 with respect to the vibration directions of the orientation control light 117. Changes in the orientation directions of the third complex 22 in a case in which the vibration directions of the orientation control light 117 are changed will be described using the above drawings. Meanwhile, in FIGS. 6A to 6C, the first complex 20a is not illustrated.

FIG. 6A is a view illustrating the orientation direction of the third complex 22 in a case in which the polarization direction control unit 120 allows the orientation control light 117 to pass through. When the orientation control light 117, which has an orientation control signal of 0 V and vibrates in the up and down direction of the paper, is allowed to pass through the polarization direction control unit 120 and enters into the reagent container 10, the third complex 22 orients with the long-axis direction oriented toward the same direction as the vibration direction of the orientation control light 117. Meanwhile, the first complex 20a and the second complex 20b, which are present in a mixed state in the blood plasma 16, have isotropic shapes, and are thus not oriented.

FIG. 6B is a view illustrating the behavior of the third complex 22 in a case in which the vibration direction of the orientation control light 117 is switched by 90 degrees, and the vibration direction of the orientation control light 117 is changed into a direction vertical to the paper. When the orientation control signal changes from 0 V to 5 V, the polarization direction control unit 120 switches the vibration direction of the orientation control light 117 from the up and down direction of the paper to the direction vertical to the paper. When the orientation control light 117, which vibrates in a direction vertical to the paper, enters into the reagent container 10, the third complex 22 rotates so that the long-axis direction becomes parallel to the vibration direction of the orientation control light 117. Meanwhile, the first complex 20a and the second complex 20b, which are present in a mixed state in the blood plasma 16, have isotropic shapes, and thus do not rotate.

FIG. 6C is a view illustrating the orientation direction of the third complex 22 in a case in which a signal of 5 V is outputted from FG 122, and the polarization direction control unit 120 changes the vibration direction of the orientation control light 117 by 90 degrees. Even in this case, the third complex 22 orients with the long-axis direction oriented toward the same direction as the vibration direction of the orientation control light 117. Meanwhile, the first complex 20a and the second complex 20b, which are present in a mixed state in the blood plasma 16, have isotropic shapes, and are thus not oriented.

As described above, the bio-molecule detecting device 100 can switch the vibration direction of the orientation control light 117, that is, switch the orientation direction of the third complex 22 in two directions having a 90-degree angular difference by outputting an orientation control signal from FG 122 so as to switch the orientation of the half-wavelength plate included in the polarization direction control unit 120.

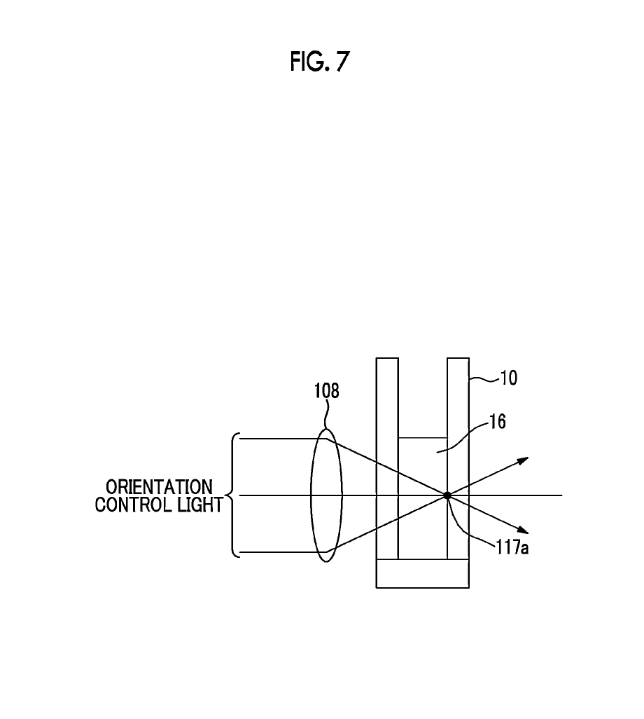

FIG. 7 is a view illustrating the location of the focus of the orientation control light 117. The orientation control light 117 enters into a lens 108, and is focused at a focus 117a in an interface between the blood plasma 16 and an inner wall surface 10a of the reagent container 10. At the location of the focus of the orientation control light, the orientation control light orients the third complexes 22 with the strongest force. Therefore, when the orientation control light enters as illustrated in FIGS. 6A to 6C, the third complexes 22 can be more efficiently oriented while the orientation control light 117 presses the third complexes to the inner wall surface 10a at the location of the focus 117a. The orientation direction of the third complexes 22 can be changed at the location of the focus 117a by rotationally moving the vibration direction of the orientation control light 117. Meanwhile, in FIGS. 6A to 6C, the second complex 20b and the third complex 22 were illustrated at locations away from the inner wall surface 10a in order to make the description easier. Meanwhile, the reagent holding unit does not necessarily have a quadrangular prism-like shape, and the same effect can be obtained as long as the reagent holding unit has a flat plane at least in some part. That is, when the orientation control light is radiated so as to be focused at an interface between the flat plane and a solution, the third complexes are pressed to the flat plane and oriented without moving aside so as to deviate from the orientation control light.

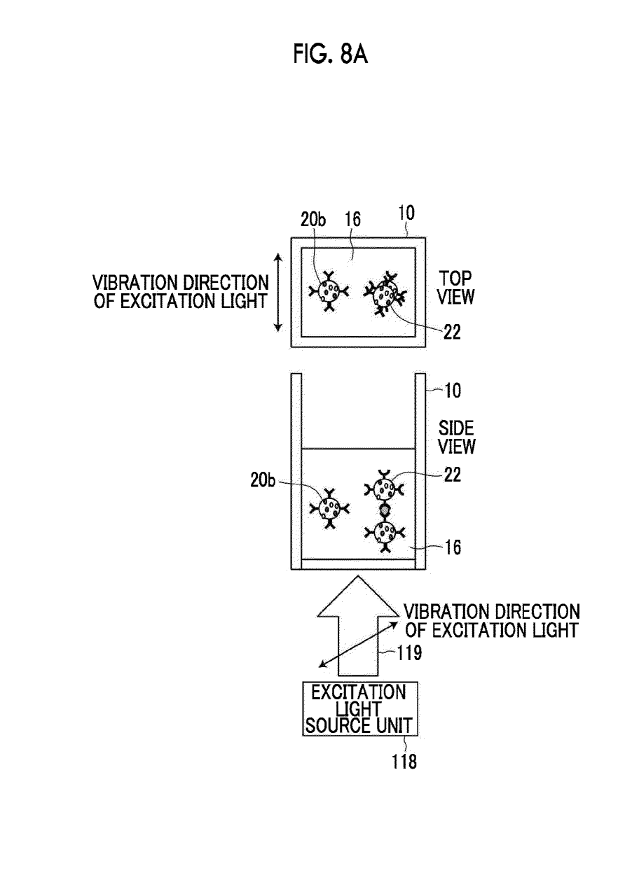

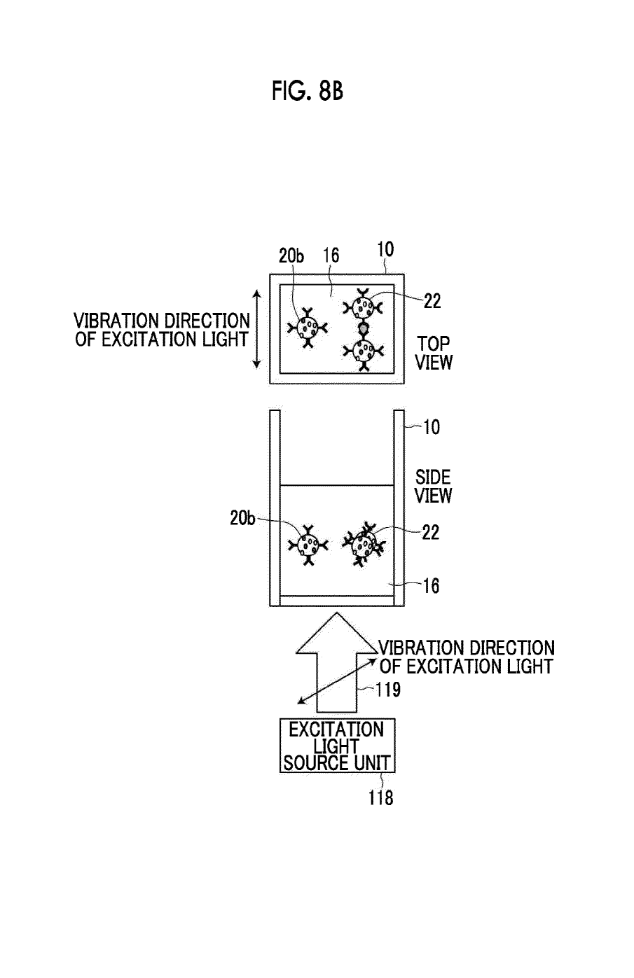

FIGS. 8A and 8B are view illustrating the vibration directions of the excitation light 119 with respect to the orientation direction of the third complex 22.

FIG. 8A is a view illustrating the relationship between the orientation direction of the third complex and the vibration direction of the excitation light in a case in which the polarization direction control unit allows the orientation control light to pass through. With respect to the side view of the reagent container 10 illustrated in FIG. 8A, the excitation light 119 travels toward the above of the paper while vibrating in a direction vertical to the paper, and enters into the blood plasma 16. With respect to the top view of the reagent container 10 illustrated in FIG. 8A, the vibration direction of the excitation light 119 becomes the up and down direction of the paper. In this case, the relationship between the vibration direction of the excitation light 119 and the orientation direction of the third complex 22 becomes the same as in FIG. 3B. Therefore, the intensity of the electric field generated by the surface plasmon resonance between the excitation light 119 and the third complex 22 becomes the same as in FIG. 3B.

FIG. 8B is a view illustrating the relationship between the orientation direction of the third complex and the vibration direction of the excitation light in a case in which the vibration direction of the orientation control light is switched by 90 degrees. Even in FIG. 8B, with respect to the side view of the reagent container 10, the excitation light 119 travels toward the above of the paper while vibrating in a direction vertical to the paper, and enters into the blood plasma 16. Even with respect to the top view of the reagent container 10 illustrated in FIG. 8B, the vibration direction of the excitation light 119 becomes the up and down direction of the paper. In this case, the relationship between the vibration direction of the excitation light 119 and the orientation direction of the third complex 22 becomes the same as in FIG. 3C. Therefore, the intensity of the electric field generated by the surface plasmon resonance between the excitation light 119 and the third complex 22 becomes the same as in FIG. 3C. Therefore, when the orientation control signal changes from 0 V to 5 V, the amount of fluorescence generated from the third complex 22 increases. Meanwhile, the amount of fluorescence generated from the first complex 20a and the second complex 20b is constant regardless of the orientation control signals. Meanwhile, even in FIGS. 8A and 8B, the second complex 20b and the third complex 22 were illustrated at locations away from the inner wall surface 10a in order to make the description easier.

As such, the bio-molecule detecting device 100 changes the orientation direction of the third complexes 22 in synchronization with the orientation control signal, and changes the fluorescence intensity generated from the third complexes 22 in synchronization with the orientation control signal. When the cycle of the reference signal inputted to the lock-in amplifier 127 is made to be the same as that of the orientation control signal, the extent of contribution of fluorescence generated from the third complexes 22 can be detected from the fluorescence generated from the entire blood plasma 16.

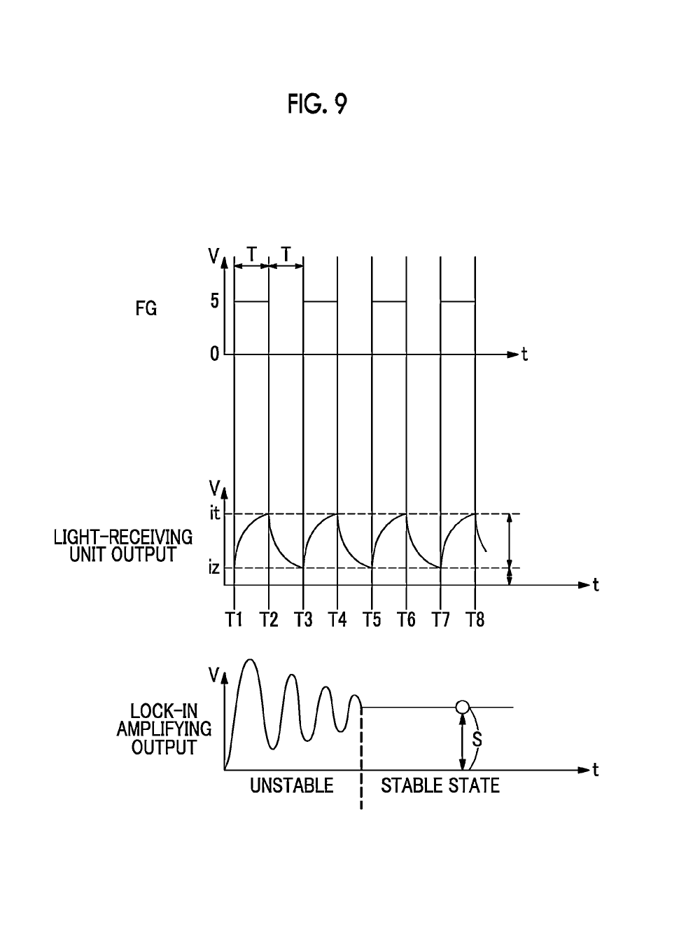

Examples of the orientation control signal outputted by FG 122 during measurement, the light-receiving unit output outputted by the light-receiving unit 124 during measurement and the lock-in amplifying output outputted by the lock-in amplifier 127 during measurement are illustrated in FIG. 9. Meanwhile, herein, in order to facilitate the description, for the light-receiving unit output and the lock-in amplifying output, the graphs are schematically illustrated.

The orientation control signal outputted from FG 122 becomes 0 V before measurement. The orientation control signal is a square wave having a cycle of 2T, which outputs a signal of 5 V in a period of 0 (second) to T (seconds), and outputs a signal of 0 V in a period of T (seconds) to 2T (seconds).

The bio-molecule detecting device 100 set the orientation control signal to 5 V at a time T1, and radiates the excitation light to the reagent container 10. When the orientation control signal is set to 5 V, the fluorescent direction control unit 120 switches the vibration direction of the orientation control light 117.

When the excitation light 119 is radiated on the blood plasma 16 at the time T1, the light-receiving unit output outputs a value of iz. The light-receiving unit output iz is the sum of fluorescence generated by the fluorescent molecules 14 included in the first complexes 20a, the second complexes 20b and the third complexes 22, all of which are included in the blood plasma 16.

Following the switching of the vibration direction of the orientation control light 117 at the time T1, the orientation direction of the third complexes 22 is switched, and the electric field between two gold nanoparticles 8 included in the third complexes 22 is intensified. Following the intensification of the electric field between two gold nanoparticles 8 included in the third complexes 22, the light-receiving unit output also increases from iz. When the orientation directions of all the third complexes 22 in the blood plasma 16 are completely switched, the light-receiving unit output is saturated at a value of it.

The orientation control signal becomes 0 V after the output of 5 V has continued for T seconds. The T seconds include at least a time period or longer, during which the orientation directions of all the third complexes 22 are completely switched, that is, a time period or longer, during which the light-receiving unit output is saturated at a value of it. The orientations of all the third complexes 22 are completely switched, and, when a time T2 is reached, the orientation control signal changes from 5 V to 0 V. When the orientation control signal changes from 5 V to 0 V, the orientation direction of the third complexes 22 is switched, and the electric field between two gold nanoparticles 8 included in the third complexes 22 is weakened. Therefore, the light-receiving unit output gradually decreases and becomes iz.

When a time period T elapses from the time T2, and the orientation control signal becomes 5 V at a time T3 again, the light-receiving unit output also increases, and is saturated at the value of it. Here, the time period during which the orientation control signal is set to 0 V was set to T seconds, similarly to the time period during which the orientation control signal was set to 5 V. This is because the time necessary to completely switch the orientation directions of the third complexes 22 in the blood plasma 16 under a condition that the output of the orientation control light 117 is constant is almost the same between a case in which the orientation control signal is set to 5 V from 0 V and a case in which the orientation control signal is set to 0 V from 5 V.

When the time period T elapses from the time T3, and the orientation control signal becomes 0 V at a time T4, the light-receiving unit output also decreases, and becomes the value of iz. Meanwhile, since a cycle of the orientation control signal is 2T, T4−T3=T3−T2=T2−T1=T. That is, the light-receiving unit output becomes a cyclic output that, similarly to the orientation control signal, the values repeatedly increase and decrease at the cycle of 2T.

The lock-in amplifier 127 detects a component that is synchronized to the reference signal from the inputted signal and increases and decreases. In the bio-molecule detecting device 100, a signal having the same cycle as the orientation control signal is inputted to the lock-in amplifier 127 as the reference signal. That is, the lock-in amplifier 127 detects a component synchronized to the orientation control signal from the light-receiving unit output. The light-receiving unit output is a cyclic signal having a 2T cycle, similarly to the orientation control signal, but what contributes to the cyclic component of the light-receiving unit output is the fluorescent molecule 14 associated with the third complex 22 oriented by the orientation control signals. Therefore, when a component synchronized with the orientation control signal is detected using the lock-in amplifier 127, the extent of contribution by the third complexes 22 can be detected from the light-receiving unit output. The lock-in amplifying output is originally an output of which repetition of increase and decrease is unstable, but gradually converges to a value of S. The value S is a light-receiving unit output based on the fluorescence generated by the fluorescent molecules 14 associated with all the third complexes 22 in the blood plasma 16.

CPU 132 computes a concentration C of the detection subject substance from the lock-in amplifying output S. Specifically, CPU obtains the concentration using the following formula (I).

C=f(S) (1)

Here, f(S) refers to a calibration curve function. The bio-molecule detecting device 100 has a different calibration curve function for each of the measurement items in advance, and converts a measured value S into a diagnosed value C. CPU 132 outputs the obtained diagnosed value C to the display unit 102.

As described above, the bio-molecule detecting device 100 according to Embodiment 1 of the invention was configured so that the orientation directions of the third complexes 22 present in the blood plasma 16 can be switched by switching the vibration direction of the orientation control light 117. The orientation directions of the third complexes 22 by the orientation control light 117 are two directions having a large difference in the intensity of the electric field generated between two gold nanoparticles 8 included in the third complexes 22, which is caused by the surface plasmon resonance. Therefore, the intensity of fluorescence generated from the third complexes 22 significantly differs due to the change in the orientation direction of the third complexes 22. In addition, since surface plasmon resonance is caused while cyclically changing the orientation direction of the third complexes 22, and the component synchronized with the cycle, at which the orientation direction of the third complexes changes, is detected from the amount of all the fluorescence generated from the blood plasma 16 using the lock-in amplifier 127, the extent of contribution of fluorescence associated with the third complexes oriented by the orientation control light 117 can be computed, and the concentration of the detection subject substance can be accurately measured with a simple configuration.

In addition, in the above configuration, since the bio-molecule detecting device 100 controls the orientations of all the third complexes 22 in the same direction using an external force generated by the orientation control light 117, high-sensitivity measurement is possible compared with a case in which measurement is made using a random movement called Brownian motion.

In addition, the time interval when switching the orientation control signal between 5 V and 0 V is desirably changed based on the mass or volume of the third complex 22, the viscosity of a solvent, the temperature of a solution, and the like. That is, the time period necessary for the orientation direction of the third complexes 22 to begin to change by the switching of the vibration direction of the orientation control light and is completely switched is determined by the volume of the third complex 22, the viscosity of a solvent, the temperature of a solution, the degree of ease for the third complex 22 to rotate in the solution, and the like. For example, in a case in which the third complexes 22 cannot be easily rotated in the solution, such as a case in which the viscosity of a specimen is high, since the time period necessary for the orientation direction of the third complexes 22 to completely switch becomes long, it is desirable to extend the period, during which the orientation control signal is set to 5 V or 0 V, long enough for the orientation direction of the third complexes 22 to completely switch. Here, the time period necessary for the orientation direction of the third complexes 22 to completely switch can be determined based on the light-receiving unit output. For example, in FIG. 9, a time period necessary for the orientation direction of the third complexes 22 to completely switch can be obtained by subtracting the time T2, at which the light-receiving unit output becomes the maximum value, by the time T1, at which the orientation control signal is first set to 5 V, that is, carrying out T2−T1.

In addition, in the embodiment, a laser having a wavelength of 909 nm and an output of 700 mW was used as the orientation control light 117, but the orientation control light 117 is not limited to the above laser. The wavelength and output intensity of the orientation control light 117 are desirably determined based on the volume, mass and the like of the third complex 22, and the degree of ease for rotating in a solution, which depends on the above parameters. The wavelength of the orientation control light 117 is not limited as long as there is no influence on the fluorescence measurement of the third complex 22. In addition, the output of the orientation control light 117 is desirably set to an output at which there is no adverse influence on complexes, such as the third complex 22. In addition, the orientation control light 117 is not necessarily an output that orients the third complex. The third complex and the sixth complex are in the Brownian motion in a solution, the intensities of fluorescence generated change even in a case in which the orientation direction is not switched. When the orientation control light 117, which is not intense enough to orient the third complex or the sixth complex, is radiated, the third complex or the sixth complex is hindered to make the Brownian motion.

In addition, in the embodiment, light having a wavelength of 635 nm and an output of 10 mW was used as the excitation light 119, but the light used as the excitation light 119 is not limited to the above light. The wavelength of the excitation light 119 is not limited as long as surface plasmon resonance is caused between the metal nanoparticles 8, but light having a wavelength band, in which the fluorescent molecule 14 is not directly excited, is desirably used. In addition, the output of the excitation light 119 is not limited as long as the surface plasmon resonance is caused between the gold nanoparticles 8, and the fluorescence generated by a generated electric field becomes intense enough to be detectable using the light-receiving unit 124. Meanwhile, the output is desirably an output having no adverse influence on the third complex 22 and the like. In addition, the excitation light source unit 118 may be configured by combining a lamp and an interference filter.

Meanwhile, in Embodiment 1, the first complex 20a has a structure in which the first antibodies 12a, the fluorescent molecules 14 and BSA 24 evenly bond to the entire surface of the metal quenching prevention film, but the first complex does not necessarily have the above structure. In addition, the second complex also does not necessarily have the structure described in Embodiment 1. Similarly, the third complex generated by bonding the first complex and the second complex to the detection subject substance does not necessarily have the structure described in Embodiment 1.

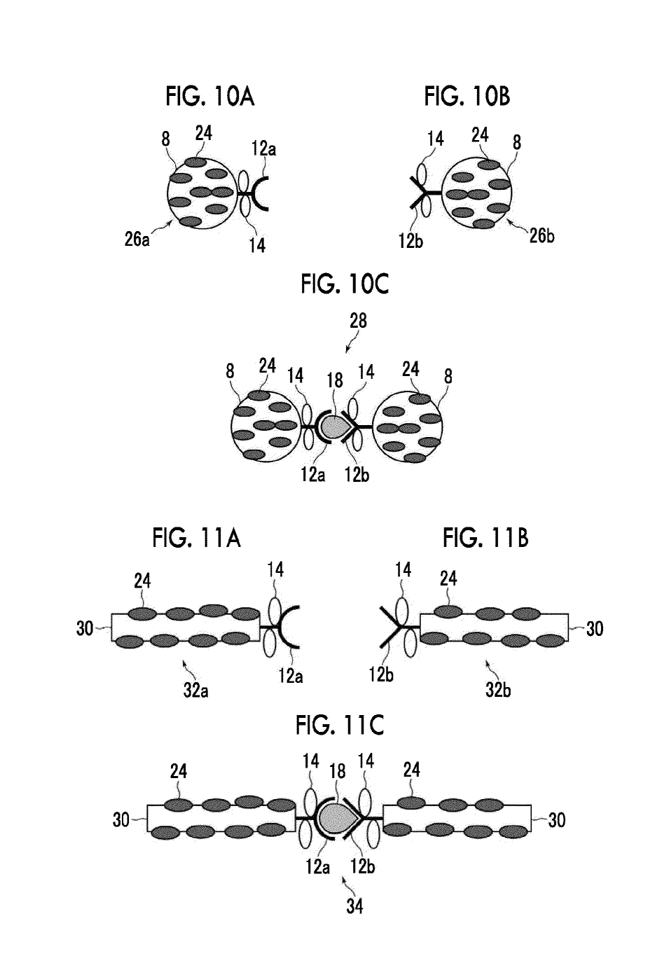

FIG. 10A is a schematic view illustrating another structure of the first complex. A first complex 26a is configured of BSA 24 evenly bonded to the entire gold nanoparticle 8, the first antibody 12a solely bonded to the gold nanoparticle 8, and a plurality of fluorescent molecules 14 bonded to the first antibody 12a. An antibody labeled with a fluorescent molecule as described above is called a fluorescent pigment-labeled antibody. The differences of the first complex 26a from the first complex 20a are that one first antibody 12a is bonded to the gold nanoparticle 8, the fluorescent molecules 14 are bonded to the first antibody 12a, and the metal quenching prevention film is not provided on the gold nanoparticle 8. Since the distance between the gold nanoparticle and the fluorescent pigment can be separated approximately several nm to 15 nm by labeling the antibodies using the fluorescent pigment, metal quenching is prevented in some part of the fluorescent pigment labeled on the antibody, and there is a merit that it is not necessary to use a particle having a special structure, such as the SiO₂ shell.

In order to produce the above structure, when the gold nanoparticle 8 and the fluorescent pigment-labeled antibody are bonded, almost the same number of the gold nanoparticles 8 and the fluorescent pigment-labeled antibodies are mixed. Then, only one fluorescent pigment-labeled antibody is bonded to the gold nanoparticle 8.

FIG. 10B is a schematic view illustrating another structure of the second complex. A second complex 26b is configured of the gold nanoparticle 8, BSA 24 evenly bonded to the gold nanoparticle 8, the second antibody 12b solely bonded to the gold nanoparticle 8, and a plurality of fluorescent molecules 14 bonded to the second antibody 12b. The differences of the first complex 26b from the second complex 20b are that one second antibody 12b is bonded to the gold nanoparticle 8, the fluorescent molecules 14 are bonded to the second antibody 12b, and the metal quenching prevention film is not provided on the metal nanoparticle 8.

The first complex 26a and the second complex 26b can be produced in the same order as the first complex 20a when a fluorescent labeled antibody is used instead of an ordinary antibody, and 20-PN-20 manufactured by Nanoparts, Inc., which is an avidin-labeled gold nanoparticle having a diameter of 20 nm, is used as the gold nanoparticle 8. An Alexa protein labeling kit manufactured by Molecular Probes is used as the fluorescent labeled antibody.

FIG. 10C is a schematic view illustrating another structure of the third complex. When the first complex 26a and the second complex 26b bond to the antigen 18, the third complex 28 is generated. Since the fluorescent molecules 14 in the first complex 26a and the second complex 26b are bonded to the first antibody 12a and the second antibody 12b respectively, in the third complex 28, the fluorescent molecules 14 are present only between the gold nanoparticle 8 included in the first complex 26a and the gold nanoparticle 8 included in the second complex 26b.

The absolute amount of the fluorescent molecules 14 decreases in the third complex 28 having the above structure compared with the third complex 22. Therefore, the amount of fluorescence generated by the single third complex also decreases. However, the intensity of the electric field generated by surface plasmon resonance changes mainly between the gold nanoparticle 8 included in the first complex and the gold nanoparticle 8 included in the second complex due to the change in the orientation direction of the third complex as illustrated in FIGS. 3B and 3C. That is, the fluorescent molecules 14 included in the third complex 28 are present only at locations at which the intensity of the electric field changes by the change in the orientation direction of the third complex 28. Therefore, when a measurement is made in the same manner as in Embodiment 1 using the first complex 26a and the second complex 26b having the above structures, only fluorescence having an intensity reflecting the intensity of the electric field generated by surface plasmon resonance is generated form the third complex 28. That is, since the change in the orientation direction of the third complex 28 is more accurately reflected in the intensity of fluorescence, the accuracy of measurement can be improved.

In addition, FIGS. 11A and 11B illustrate another examples of the first complex and the second complex forming the third complex, in which the fluorescent molecules are present only at locations at which the intensity of the electric field changes by the changes in the orientation direction. FIG. 11A is a schematic view illustrating another structure of the first complex. The first complex 32a is configured of a gold nanorod 30, BSA 24 evenly bonded to the entire surface of the gold nanorod 30, the first antibody 12a solely bonded to the gold nanorod 30, and a plurality of the fluorescent molecules 14 bonded to the first antibody 12a. The difference from the first complex 32a from the first complex 26a is that the gold nanorod 30 is used instead of the gold nanoparticle 8. The gold nanorod 30 is a gold nanoparticle having a cylindrical shape. A nanorod having a short axis of 10 nm and a long axis of approximately 50 nm is used as the gold nanorod 30.

FIG. 11B is a schematic view illustrating another structure of the second complex. The second complex 32b is configured of the gold nanorod 30, BSA 24 evenly bonded to the entire surface of the gold nanorod 30, the second antibody 12b solely bonded to the gold nanorod 30, and a plurality of the fluorescent molecules 14 bonded to the second antibody 12b. The difference from the second complex 32b from the second complex 26b is that the gold nanorod 30 is used instead of the gold nanoparticle 8.

The first complex 32a and the second complex 32b can be produced in the same order as the first complex 20a when a fluorescent labeled antibody is used instead of an ordinary antibody, and an avidin-labeled gold nanorod is used as the gold nanoparticle.

FIG. 11C is a schematic view illustrating another structure of the third complex. When the first complex 32a and the second complex 32b are bonded to the antigen 18, the third complex 34 is generated. Since the fluorescent molecules 14 in the first complex 32a and the second complex 32b are bonded to the first antibody 12a and the second antibody 12b respectively, in the third complex 32, the fluorescent molecules 14 are present only between the gold nanoparticle 8 included in the first complex 32a and the gold nanoparticle 8 included in the second complex 32b. Even when a measurement is made in the same manner as in Embodiment 1 using the first complex 32a and the second complex 32b having the above structures, similarly to a case in which a measurement is made using the first complex 28a and the second complex 28b, the accuracy of measurement can be improved. In addition, even when the first complex 32a and the second complex 32b having the above structures are used, since the fluorescent molecule 14 is sufficiently away from the surface of the gold nanorod 30, metal quenching can be prevented without providing the metal quenching prevention film and the like in the gold nanorod 30.

### Embodiment 2

In Embodiment 2, four kinds of complexes are used, and two kinds of specific antigens, which are detection subject substances, are detected in a homogeneous solution. Since two out of the four kinds of complexes are the same as the first complex 20a and the second complex 20b described in Embodiment 1, they will not be described. A fourth complex and a fifth complex, which are the remaining two kinds of complexes used in the present embodiment, will be described.

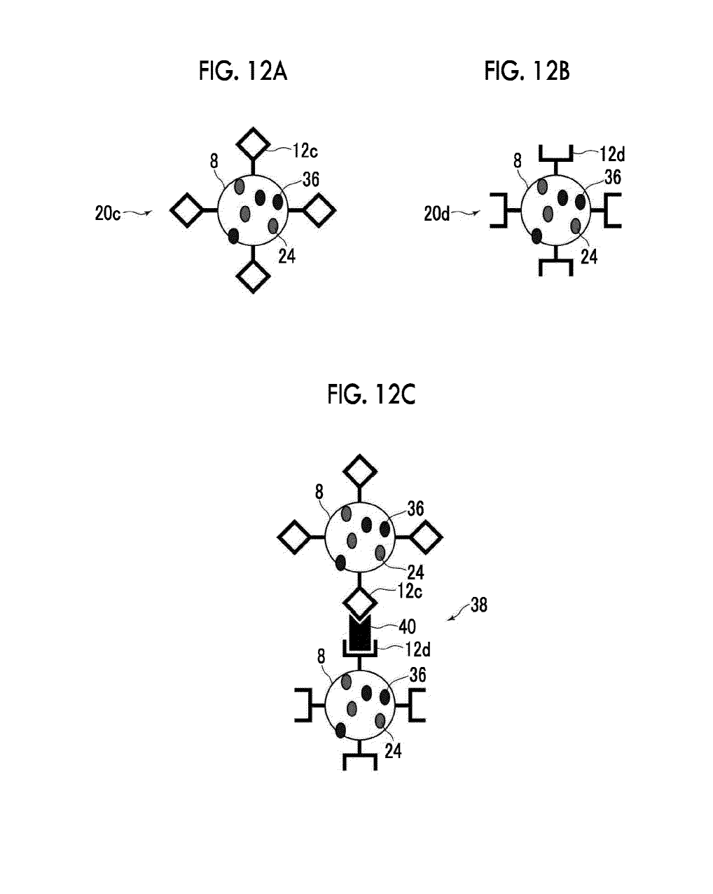

FIG. 12A is a schematic view of the fourth complex 20c. The fourth complex 20c is formed by covering the entire surface of the substantially spherical gold nanoparticle 8 having a diameter of 20 nm with a 10 nm-thick metal quenching prevention film made of SiO₂, and bonding a plurality of third antibodies 12c, a plurality of fluorescent molecules 36, and a plurality of BSA 24 to the surface of the metal quenching prevention film. The third antibodies 12c, the fluorescent molecules 36 and BSA 24 are evenly bonded to the entire surface of the metal quenching prevention film included in the fourth complex 20c. That is, the fourth complex 20c has different kinds of antibodies and fluorescent molecules compared with the first complex 20a.

FIG. 12B is a schematic view of a fifth complex 20d used in Embodiment 2 of the invention. The fifth complex 20d is formed by covering the entire surface of the gold nanoparticle 8 having a diameter of 20 nm with a 10 nm-thick metal quenching prevention film made of SiO₂, and bonding a plurality of fourth antibodies 12d, a plurality of the fluorescent molecules 36, and a plurality of BSA 24 to the surface of the metal quenching prevention film. The fourth antibodies 12d, the fluorescent molecules 36 and BSA 24 are evenly bonded to the entire surface of the metal quenching prevention film included in the fifth complex 20d. That is, the fifth complex 20d has different kinds of antibodies and fluorescent molecules compared with the first complex 20a. As such, the fourth complex 20c and the fifth complex 20d have the same isotropic shape as those of the first complex 20a and the second complex 20b.

The third antibody 12c and the fourth antibody 12d are antibodies that react with specific portions of an antigen, which is a detection subject substance, and bond to the respectively different locations. In the embodiment, MAB4128 (Human CEACAM-5 Antibody) manufactured by R&D SYSTEMS Inc. was used as the third antibody 12c, and MAB4128 (Human CEACAM-5) manufactured by R&D SYSTEMS Inc. was used as the fourth antibody 12d. In the embodiment, two kinds of antigens, which are ErbB2/Her2 protein and CEA protein, in the blood plasma will be detected as detection subject substances. Hereinafter, ErbB2/Her2 protein, which is the detection subject substance, will be called the antigen 18, and CEA protein, which is the detection subject substance, will be called an antigen 40. The first antibody 12a, the second antibody 12b, the third antibody 12c and the fourth antibody 12d used in the embodiment are antibodies for detecting antigens using a so-called sandwich method. Therefore, the antibodies are bonded to the antigen after recognized using respectively different epitopes. That is, the first antibody 12a and the second antibody 12b specifically bond to the respectively different locations of the antigen 18. The third antibody 12c and the fourth antibody 12d specifically bond to the respectively different locations of the antigen 40. In addition, the first antibody 12a and the second antibody 12b, and the third antibody 12c and the fourth antibody 12d bond to far locations in the steric structure of the antigen so as to prevent the antibodies from causing a steric hindrance to each other. Meanwhile, the third antibody 12c and the fourth antibody 12d in FIGS. 12A and 12B are illustrated using respectively different recess shapes, thereby indicating that the antibodies bond to different locations of the antigen, but the third antibody 12c and the fourth antibody 12d do not necessarily have such shapes.

Alexa Fluor 647 (product name of Molecular Probes) was used as the fluorescent molecule 36. Alexa Fluor 647 has a peak at a wavelength of approximately 670 nm, and emanates fluorescence having a wavelength of approximately 620 nm to 750 nm. The fourth complex 20c and the fifth complex 20d have the fluorescent molecules 36 of the same type. The fourth complex 20c and the fifth complex 20d are produced in the same order as the first complex 20a.

FIG. 12C is a view illustrating a case in which both the fourth complex 20c and the fifth complex 20d bond to the antigen 40. Hereinafter, a complex, in which both the fourth complex 20c and the fifth complex 20d bond to the antigen 40 as illustrated in the drawing, will be called a sixth complex 38. In FIG. 12C, the antigen 40 is illustrated to have protrusion and recess shapes matching the respective protrusion and recess portions of the third antibody 12c and the fourth antibody 12d in order to illustrate that the third antibody 12c and the fourth antibody 12d specifically bond to respectively different locations of the antigen 40. Therefore, the actual antigen 40 does not always have such shapes.

Since the third antibody 12c and the fourth antibody 12d are antibodies for carrying out the sandwich method on the antigen 40, in a case in which both the fourth complex 20c and the fifth complex 20d bond to the antigen 40, the fourth complex 20c and the fifth complex 20d bond to locations where the complexes face each other with the antigen 40 therebetween as illustrated in FIG. 12C. That is, the sixth complex 38 has a structure in which the fourth complex 20c, the antigen 40 and the fifth complex 20d are linearly arrayed. Hereinafter, in the sixth complex 38, a direction in which the fourth complex 20c, the antigen 40 and the fifth complex 20d are arrayed will be called a long-axis direction, and a vertical direction to the long-axis direction will be called a short-axis direction. In the sixth complex 38, the distance between the gold nanoparticle 8 included in the fourth complex 22c and the gold nanoparticle 8 included in the fifth complex 22d becomes approximately 30 nm.

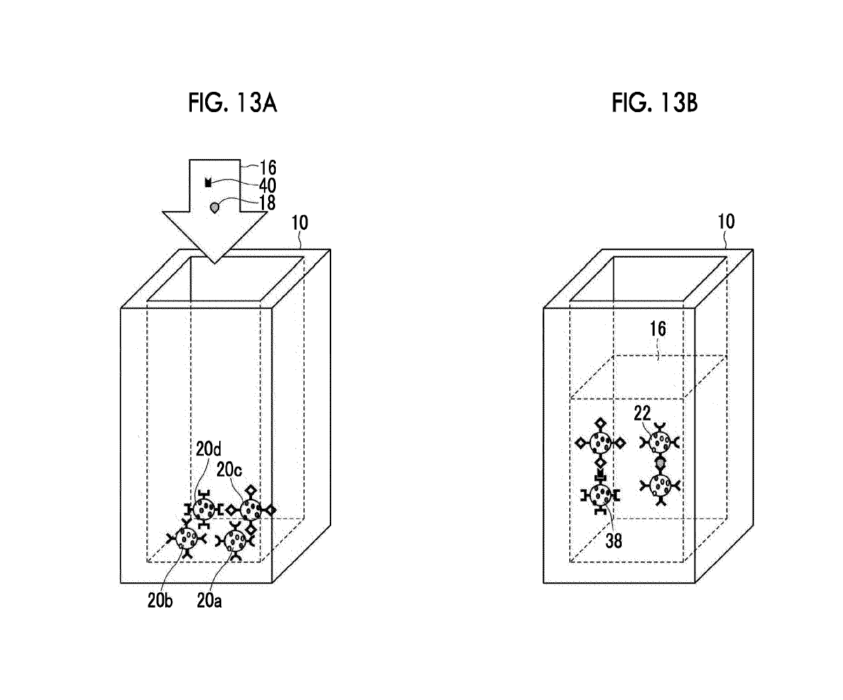

FIGS. 13A and 13b are schematic views illustrating the overview of an antigen-antibody reaction in the bio-molecule detecting device according to the embodiment. FIG. 13A illustrates a state before the antigen-antibody reaction. In FIGS. 13A and 13B, the reagent holding unit, not exposed to the outside, is illustrated using broken lines. In the reagent holding unit, a plurality of the dried first complexes 20a and a plurality of the dried second complexes 20b, the fourth complex 20c and the fifth complex 20d are fed.

Even in the embodiment, similarly to Embodiment 1, the specimen is the blood plasma 16 separated from whole blood. When the blood plasma 16 is injected into the reagent container 10, and stirred, in a case in which the antigen 18 that specifically bonds to the first antibody 12a and the second antibody 12b is present in the blood plasma 16, an antigen-antibody reaction is caused between the first antibody 12a and the second antibody 12b and the antigen 18, and the third complex 22 is formed as illustrated in FIG. 2B. In addition, in a case in which the antigen 40 that specifically bonds to the third antibody 12c and the fourth antibody 12d is present in the blood plasma 16, an antigen-antibody reaction is caused between the third antibody 12c and the fourth antibody 12d and the antigen 40, and the sixth complex 38 is formed as illustrated in FIG. 2B.

While not illustrated, since a sufficiently large amount of the first complex 20a, the second complex 20b, the fourth complex 20c and the fifth complex 20d are fed with respect to the antigen 18 and the antigen 40, some of the first complex 20a, the second complex 20b, the fourth complex 20c and the fifth complex 20d remain in the blood plasma 16 without causing an antigen-antibody reaction. That is, in the blood plasma 16, in which the first complex 20a, the second complex 20b, the fourth complex 20c and the fifth complex 20d are mixed, the first complex 20a, the second complex 20b, the third complex 22, the fourth complex 20c, the fifth complex 20d and the sixth complex 38 are present in a mixed state.

Meanwhile, components other than the antigen 18 and the antigen 40 are also present in the blood plasma 16, but components other than the antigen 18 and the antigen 40 will not be illustrated in FIGS. 13A and 13B in order to simplify the description.

A bio-molecule detecting device 200 according to the embodiment carries out the detection and quantity determination of the antigen 18 and the antigen 40 by radiating excitation light on the blood plasma 16, which is a liquid phase so that the first complex 20a, the second complex 20b, the third complex 22, the fourth complex 20c, the fifth complex 20d and the sixth complex 38 are present in a mixed state, causing surface plasmon resonance between the excitation light and the gold nanoparticle 8, emitting the light of the fluorescent molecule 14 and the fluorescent molecule 36 using an electric filed generated by the surface plasmon resonance, and measuring fluorescence generated from the inside of the blood plasma 16.

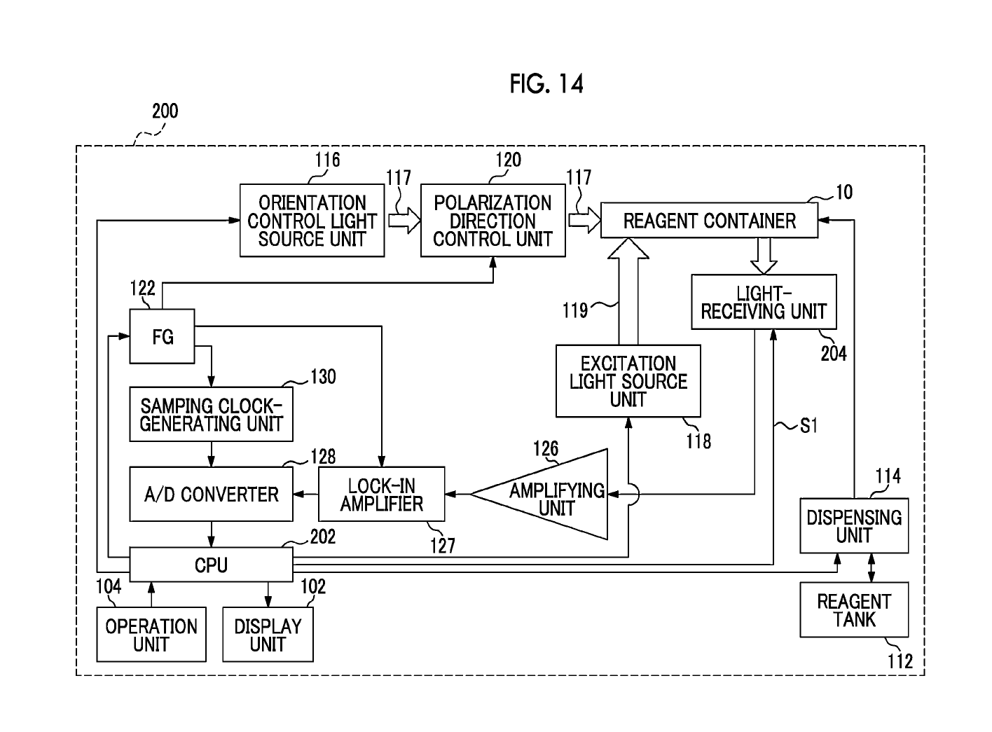

FIG. 14 is a function block diagram for explaining the principal configuration of the bio-molecule detecting device 200. Meanwhile, the same configuration element as in Embodiment 1 will be given the same reference numeral, and will not be described. Compared with the bio-molecule detecting device 100, in the bio-molecule detecting device 200, CPU 202 and a light-receiving unit 204 are mainly different.

CPU 202 carries out the computation of digital data sent from the A/D converter 128, and outputs a result to the display unit 102. In addition, CPU 202 receives an input from the user input unit 104, instructs and orders the operation of the orientation control light source unit 116, the excitation light source unit 118, the dispensing unit 114, FG 122 and the light-receiving unit 204. Specifically, CPU carries out the ordering of the ON and OFF of the orientation control light source unit 116 and the excitation light source unit 118, the ordering of designating a reagent to use and starting a dispensing operation with respect to the dispensing unit 114, the ordering of instructing and outputting a signal waveform to output with respect to FG 122, and the ordering of switching filters with respect to the light-receiving unit 204.

The light-receiving unit 204 is a light-receiving unit that detects fluorescence generated from the fluorescent molecules in the reagent container 10. The light-receiving unit 204 is configured to receive an order (S1) from CPU 208 and separate the fluorescence of the fluorescent molecules 14 and the fluorescent molecules 36, thereby receiving light.

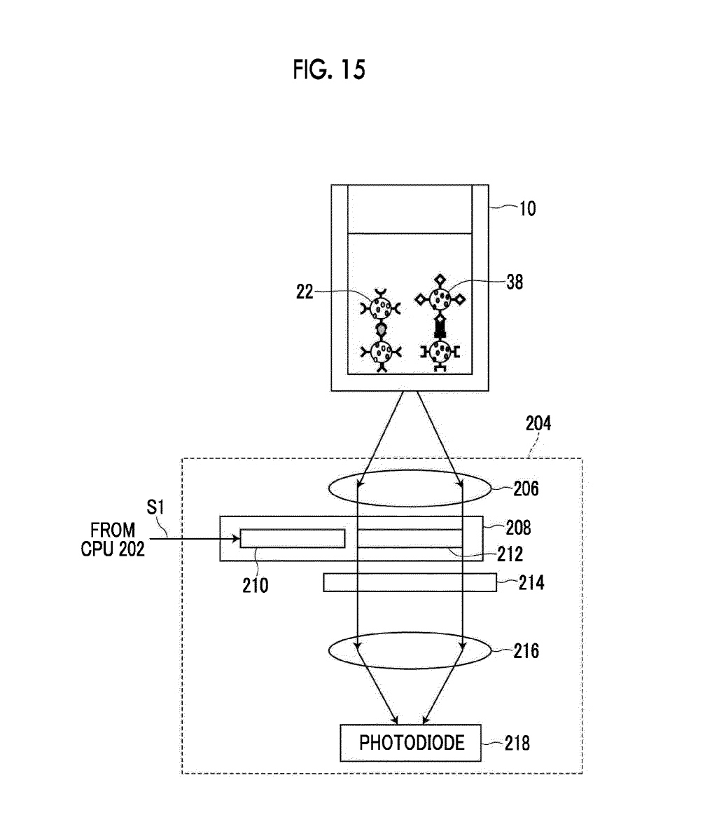

The configuration of the light-receiving unit 204 will be specifically described using FIG. 15. FIG. 15 is a schematic view expressing the detailed configuration of the light-receiving unit 204 in the bio-molecule detecting device 200 according to Embodiment 2. The light-receiving unit 204 has a lens 206, a lens 216, a filter-switching unit 208 and a polarizer 214. The light of fluorescence generated from the fluorescent molecules 14 and the fluorescent molecules 36 in the reagent container 10 is collected using the lens 206, and entered into a photodiode 218 through the filter-switching unit 208, the polarizer 214 and the lens 216.

The filter-switching unit 208 has two kinds of filters which are a filter 210 and a filter 212. The two kinds of filters are mobile, and the filters, through which fluorescence collected using the lens 206 passes, can be switched. The filter-switching unit 208 receives the order S1 from CPU 202, and switches the filters through which fluorescence passes.

The filter 210 is a band pass filter that transmits only light having a wavelength band in which the fluorescent molecules 14 are generated. The filter 212 is a band pass filter that transmits only light having a wavelength band in which the fluorescent molecules 36 are generated. When measuring the antigen 18, the filter-switching unit 208 locates the filter 210 in the light path of fluorescence and carries out a measurement, and, when measuring the antigen 40, the filter-switching unit locates the filter 212 in the light path of fluorescence and carries out a measurement according to the order S1 from CPU 202. With the above configuration, the light-receiving unit 204 prevents light, which is not generated from the complexes including the detection subject substance, from reaching the photodiode 218. Meanwhile, it is not always necessary to use the filter, and, for example, a diffraction lattice or the like may be used to separate light.

The polarizer 214 transmits only light polarized in the same direction as the polarization direction of the excitation light 119. Since the polarization direction of the excitation light 119 scattered in the reagent container 10 or fluorescence emitted from the fluorescent molecules 14 or the fluorescent molecules 36 in the middle of the switching of the orientation direction is different from the original polarization direction of the excitation light, the excitation light or the fluorescence cannot transmit through the polarizer 146.

PD 218 is configured of an avalanche photodiode (APD), receives fluorescence collected using the lens 216, and generates charges in accordance with the intensity of the fluorescence, thereby outputting to the amplifying unit 126.

Subsequently, the measurement action of the bio-molecule detecting device 200 will be described. The measurement action of the bio-molecule detecting device 200 is basically the same as the measurement action of the bio-molecule detecting device 100 described in Embodiment 1, but is different in minutiae. Since the reason why only fluorescence generated from the complexes including the detection subject substance can be separated has been described in Embodiment 1, herein, a method for separating and detecting the third complex 22 and the sixth complex 38 respectively including two kinds of detection subject substances will be described.

First, the bio-molecule detecting device 200 determines which of the antigen 18 and the antigen 40 is first detected. This can be arbitrarily determined by a user's input operation or the like in the user input unit 104. Here, the third complex 22 including the antigen 18 is first detected. CPU 202 sends an order of instructing the use of the filter 210 to the filter-switching unit 208 in the light-receiving unit 204. The filter-switching unit 208 receives the order from CPU 202, and moves the filter 210 to a location at which light collected using the lens 206 passes. When the orientation control signal is changed to 5 V, and the excitation light 119 is radiated toward the reagent container 10, the fluorescent molecules 14 and the fluorescent molecules 36 in the solution generate fluorescence. The fluorescence generated from the fluorescent molecules 14 and the fluorescent molecules 36 are collected using the lens 206, and enters into the filter 210. Since the filter 210 transmits only light having a wavelength band in which the fluorescent molecules 14 are generated, almost all the fluorescence generated from the fluorescent molecules 36 is blocked. The light-receiving unit 204 can detect only the fluorescence generated from the fluorescent molecules 14 in the above manner.

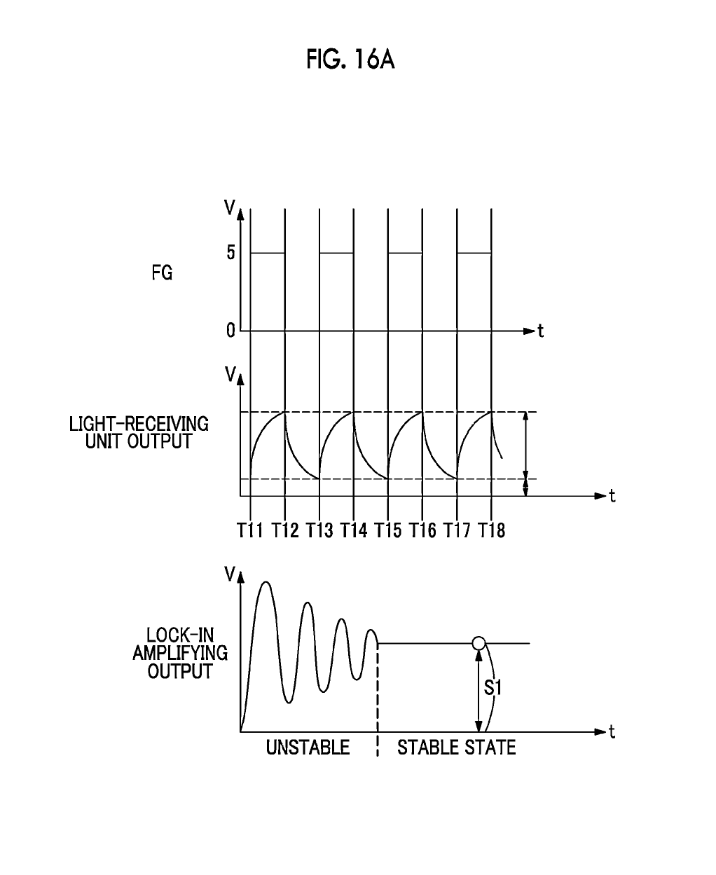

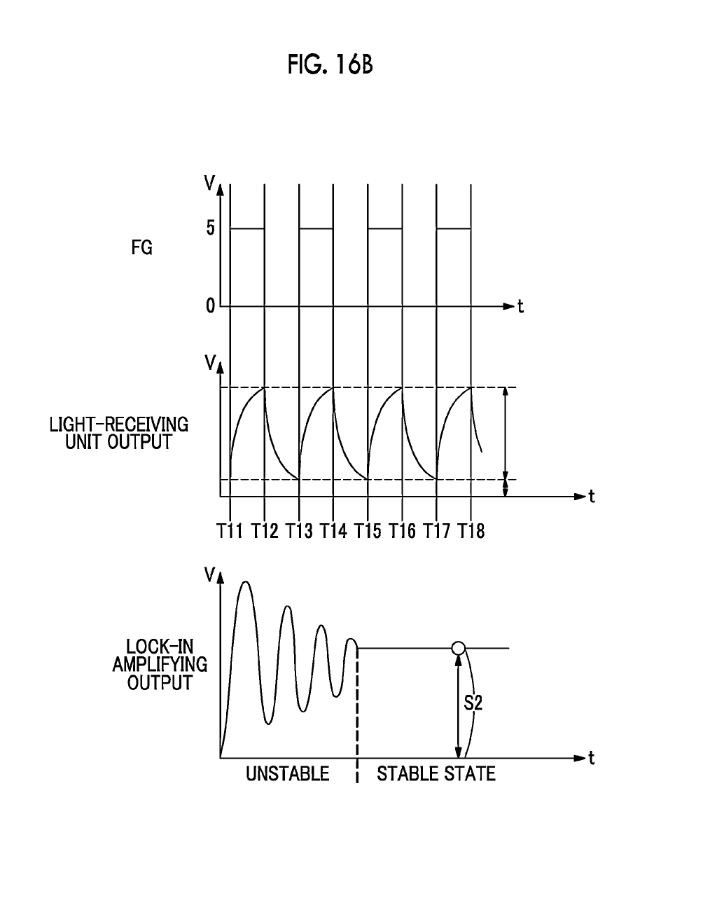

Similarly to Embodiment 1, several cycles of the orientation control signals are measured using the bio-molecule detecting device 200, and a light-receiving unit output of the result of the detection of the fluorescence generated from the fluorescent molecules 14 is illustrated in FIG. 16A. The light-receiving unit output outputs a signal having the same cycle as the orientation control signal. The lock-in amplifier detects a component synchronized to the orientation control signal in the light-receiving unit output, and outputs a value S1.

Subsequently, CPU 202 computes the concentration of the antigen 18 from the obtained value S1. Specifically, the measured value S1 is converted into a concentration C1 using the calibration curve function f1(S) in the same manner as in Embodiment 1. CPU 202 outputs the obtained concentration C1 to the display unit 102.

Next, the bio-molecule detecting device 200 carries out the measurement of the sixth complex 38 having the antigen 40. CPU 202 sends an order of instructing the use of the filter 212 to the filter-switching unit 208 in the light-receiving unit 204. The filter-switching unit 208 receives the order from CPU 202, and moves the filter 212 to a location at which light collected using the lens 206 passes. Since the filter 212 transmits only light having a wavelength band in which the fluorescent molecules 36 are generated, almost all the fluorescence generated from the fluorescent molecules 14 is blocked. The light-receiving 204 can detect only the fluorescence generated from the fluorescent molecules 36 in the above manner.

Several cycles of the orientation control signals are measured using the bio-molecule detecting device 200, and a light-receiving unit output of the result of the detection of the fluorescence generated from the fluorescent molecules 36 is illustrated in FIG. 16B. In FIGS. 16A and 16B, the graphs are schematically illustrated in order to facilitate calculation. The light-receiving unit output outputs a signal having the same cycle as the orientation control signal.

The switching timing of the orientation control signal in a case in which the sixth complex 38 is measured is different from the case in which the third complex 22 is measured. This is because the volumes and masses of the third complex 22 and the sixth complex 38 are different, and times necessary for the respective complexes to complete the orientation are different.

As illustrated in FIGS. 16A and 16B, there are differences in the maximum values and minimum values of the light-receiving unit outputs between the case in which the third complex 22 is measured and the case in which the sixth complex 38 is measured. This results from the difference in the concentration in the solution between the third complex 22 and the sixth complex 38.

Subsequently, CPU 202 obtains the concentration of the antigen 40 from the obtained value S2. Specifically, the measured value S2 is converted into a concentration C2 using the calibration curve function f2(S). CPU 202 outputs the obtained concentration C2 to the display unit 102.

As described above, according to the bio-molecule detecting device 200 according to Embodiment 2 of the invention, in addition to the configuration of the bio-molecule detecting device 100 described in Embodiment 1, four kinds of complexes and two kinds of fluorescent molecules were used as substances specifically bonding to detection subject substances, and the filter-switching unit 208 was configured to be capable of switching two kinds of filters. Therefore, only fluorescence generated from the fluorescent molecules associated with the complex including the detection subject substance can be detected by using a filter corresponding to the fluorescent molecules associated with the complex including the detection subject substance, and the concentrations of two kinds of detection subject substances included in a specimen can be accurately measured.

Meanwhile, in the embodiment, Alexa Fluor 568 and Alexa Fluor 647 were used as the fluorescent molecules, but the fluorescent molecules being used are not limited thereto. Complexes, to which a plurality of detection subject substances are specifically bonded respectively, may be labeled using respectively different fluorescent molecules, and fluorescent molecules having fluorescence wavelengths, excitation wavelengths or fluorescence service lives, which are different enough to be separated using filters, may be used.

In addition, in the embodiment, a case in which two kinds of substances are detected as the detection subject substances has been described, but more detection subject substances may be detected. Even in this case, the respective detection subject substances can be separated and detected by carrying out the sandwich method using two kinds of complexes that specifically bond to the respective detection subject substances, labeling the complexes using respectively different fluorescent molecules, separating and detecting fluorescence generated from the respective florescent molecules using filters corresponding to the respective fluorescence.

Meanwhile, since the kind of the fluorescent molecules also increases as the detection subject substance increases, and fluorescence generated from a plurality of fluorescent molecules is present in a mixed state, the detection of fluorescence generated by the target fluorescent molecules can be facilitated by using a band pass filter having a narrower passband.

The method for switching the orientation of the third complex 22 or the sixth complex 38 in the solution is not limited to a laser, and a magnetic method or an electric method may be used as long as the complexes can be oriented.

When the orientations of molecules are controlled using light, a complicated mechanism is not necessary compared with a case in which the orientations of molecules are controlled using a magnetic force or the like. For example, in order to control the orientations of molecules using a magnetic force, the respective molecules need to be magnetic, or it is necessary to prepare magnetic molecules and bond the molecules to molecules of which orientations are to be controlled, which makes the preparation for measurement troublesome.

In addition, the orientation direction of the third complex 22 or the sixth complex 38 does not necessarily need to be switched using the half-wavelength plate. For example, the orientation direction may be switched by switching the radiation direction of the orientation control light 117 using an acousto optic deflector (AOD) instead of switching the vibration direction of the orientation control light 117. In addition, in a case in which the orientation direction is switched by switching the radiation direction of the orientation control light 117, the radiation direction of the orientation control light may be switched by providing a plurality of light source units for controlling the orientation of an orientation control light.

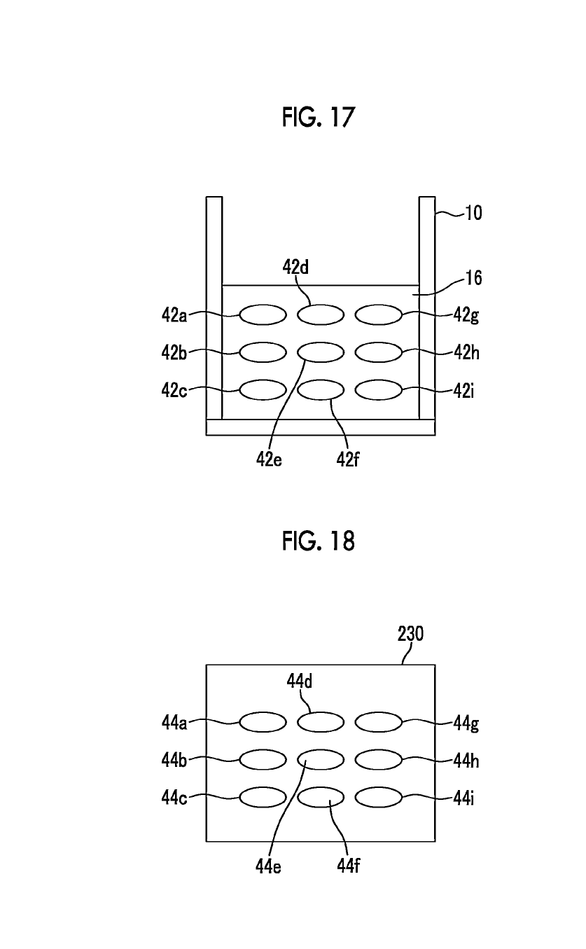

In addition, the number of the orientation control light source unit provided does not necessarily need to be one, and a plurality of orientation control light may be radiated in the same direction by providing a plurality of orientation control light source units. For example, as illustrated in FIG. 17 (the side view of the reagent container 10), a plurality of orientation control light source units may be provided so as to enter nine rays of orientation control light corresponding to 9 points of 42a to 42i respectively. When orientation control light is radiated from a plurality of locations as illustrated above, the third complexes 22 and the sixth complexes 38, which are located in the center of the polarization axis of the orientation control light, increase. Around the center of the polarization axis of the orientation control light, the third complexes 22 or the sixth complexes 36 can be most efficiently moved rotationally. Meanwhile, here, an example in which the orientation control light is entered into 9 points has been described, but the number of points into which the orientation control light is entered is not limited to 9, and may be larger or smaller than 9. As the orientation control light is squeezed more minutely, the orientation control light is desirably entered into more points. Thereby, the third complexes 22 or the sixth complexes 38 can be moved rotationally in synchronization with the orientation at a plurality of places. As a result, an unexpected change in the fluorescence intensity can be decreased, and the coefficient of variation, which is an index that indicates a relative diffusion, can be improved. Even in this case, the respective orientation control light is desirably radiated so as to be focused at an edge surface at which the reagent container 10 outgoes.

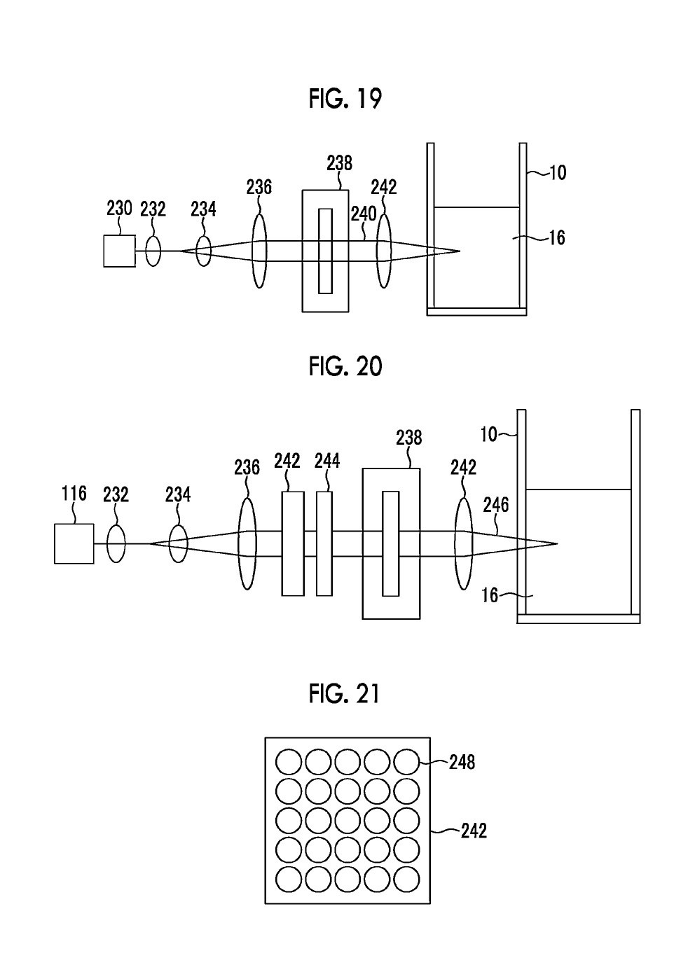

The structure of the orientation control light source unit for entering the orientation control light into multiple points in the above manner will be illustrated in FIG. 18. An orientation control light source unit 230 is a 3×3 two-dimensional laser array. The orientation control light source unit 230 emits light at 9 light-emitting points of 44a to 44i. The size of the light-emitting point is 1 μm in the vertical side and 100 μm in the horizontal side. FIG. 19 illustrates an example of an optical system in which the orientation control light source unit 230 is used. Meanwhile, in FIG. 19, components other than the optical system relating to the orientation control light are not illustrated.

A linearly polarized orientation control light 240 radiated from the orientation control light source unit 230 passes through a collimator lens 232, and becomes a parallel light ray at the focus. The orientation control light 240, which has passed through the collimator lens 232, passes through a beam expander 234 and a beam expander 236, and enters into a polarization direction control unit 238 having a half-wavelength plate. The orientation control light 240, which has passed through the beam expander 234 and the beam expander 236, is spread into parallel beams of a specific magnification. The half-wavelength plate is located on a rotary stage so as to be rotatable. Thereby, the polarization axis of the orientation control light 240 can be rotationally moved. The orientation control light 240, which has transmitted through the half-wavelength plate, is collected using a lens 242, and enters into a side surface of the reagent container 10.

In the optical system illustrated in FIG. 19, when the focal distance of the collimator lens 232 is set to 3.1 mm, and the focal distance of the lens 242 is set to 4 mm, the magnification becomes 1.29 times. Therefore, on the side surface of the reagent container 10, the size of the orientation control light 240 becomes approximately 1.3 μm×130 μm, and the pitch becomes approximately 129 μm.

FIG. 20 illustrates an example of another optical system that enters the orientation control light into multiple points. Meanwhile, even FIG. 20 does not illustrate components other than the optical system of the orientation control light. In addition, the same configuration element as in FIG. 19 will be given the same reference numeral, and will not be described.

In the optical system illustrated in FIG. 20, the orientation control light source unit 116 is the same as in Embodiment 1. An orientation control light 246 passes through a collimator lens 406, a beam expander 408 and a beam expander 410, and enters into a micro lens array 242. The micro lens array 242 has a plurality of micro lenses 248 arrayed in a lattice shape as illustrated in FIG. 21. The orientation control light 246, which has passed through the micro lens array 248, becomes a plurality of beams focused at different locations like light radiated from a plurality of light sources. The orientation control light 246 is squeezed using a pin hole array 244, passes through the polarization direction control unit 238, is collected using the lens 242, and enters into the side surface of the reagent container 10. Even when the micro lens array is used as described above, the orientation control light can be entered into multiple points.

In addition, in an optical system in which the radiation direction of the orientation control light is changed as in Embodiment 1, a plurality of steps of optical systems may be prepared in order to radiate orientation light on multiple points. For example, when three steps of the same optical systems are superimposed, the orientation control light is radiated from three orientation control light source unit and can be entered to the reagent container 10 from three points. In this manner, the orientation control light can be radiated from multiple points, and the third complexes 22 or the sixth complexes 38 can be moved rotationally at a plurality of places.

In addition, in the respective embodiments according to the invention, the vibration direction of the orientation control light 117 is switched between two orthogonal directions, but the vibration direction of the orientation control light does not necessarily need to be switched between two orthogonal directions. For example, in a case in which only the detection of the detection subject substance needs to be carried out, the third complexes 22 or the sixth complexes 38 may be orientated in a different direction so that the intensity of fluorescence generated from the third complexes 22 or the sixth complexes 38 changes.

When the vibration direction of the orientation control light 117 is switched between two orthogonal directions, the necessary time for the third complexes 22 or the sixth complexes 38 to be completely oriented becomes the maximum so that S/N becomes most favorable. Meanwhile, for example, when the angle formed by two travelling directions of the orientation control light 117 is 60 degrees, compared with a case in which the travelling directions of the orientation control light 117 are orthogonal, the time for the orientation of the third complexes 22 or the sixth complexes 38 to be completely switched becomes short, and the necessary time for measurement also becomes short. As such, as the angle formed by two travelling directions of the orientation control light 117 decreases below 90 degrees, the necessary time for the orientation of the third complexes 22 or the sixth complexes 38 to be completely switched becomes shorter, and the measurement time also becomes shorter.

Meanwhile, in the respective embodiments, a case in which one reagent container 10 is provided in the bio-molecule detecting device has been described, but the number of the reagent containers 10 does not necessarily need to be one, and the bio-molecule detecting device may be configured to provide a plurality of reagent containers in the device so that a plurality of specimens can be set. In this case, when the bio-molecule detecting device is configured to sequentially move the reagent containers to measurement locations and carry out measurements, a plurality of specimens can be automatically measured.

Meanwhile, in the respective embodiments, a measurement has been carried out using the first complexes 20a, the second complexes 20b, the fourth complexes 20c or the fifth complexes 20d, which have been generated in advance, but the first complexes 20a, the second complexes 20b, the fourth complexes 20c or the fifth complexes 20d may be generated in the reagent container 10. In this case, the user prepares gold nanoparticles, antibodies and fluorescent molecules in the respectively separate reagent tanks, the bio-molecule detecting device injects the gold nanoparticles, the antibodies, the fluorescent molecules and specimens into the reagent container 10 respectively at a time of measurement, and causes reactions.

Meanwhile, in the respective embodiments, the gold nanoparticles were used as particles that cause the surface plasmon resonance with the excitation light, but the particles does not necessarily need to be gold nanoparticles. For example, silver nanoparticles or copper nanoparticles may be used.

In addition, the orientation control light source unit 116 or the excitation light source unit 118 may be configured to be attachable and detachable so that the unit can be replaced by an appropriate unit depending on the detection subject substance, the fluorescent molecule, and the like.

The time interval of switching the orientation direction is desirably determined depending on the necessary time for the orientation of all the third complexes 22 or the sixth complexes 38 to be completely switched based on the masses or volumes of the detection subject substance, the third complex 22 and the sixth complex 38, the intensity of the external force by the orientation control unit, and the like. That is, the orientation direction is desirably switched every necessary time for the orientation of all the third complexes 22 or the sixth complexes 38 to be completely switched. When the switching time of the orientation direction is determined as described above, it becomes unnecessary to radiate the orientation control light 117 in the same direction even after all the third complexes 22 or the sixth complexes 38 are completely oriented, and the power consumption can be reduced. In addition, it becomes unnecessary to continue the measurement at an unnecessary time, and the measurement time can be reduced.

The necessary time for the orientation of all the third complexes 22 or the sixth complexes 38 to be completely switched may be obtained based on the light-receiving unit output or the A/D converter output. For example, when measurement is repeated several cycles, it is found approximately how much time is required for the respective outputs to be saturated, and therefore the time computed by arithmetically averaging the necessary times for the respective outputs to be saturated may be determined as a predetermined time interval.

Meanwhile, in the respective embodiments of the invention, a case in which the blood plasma separated from whole blood was used as the specimen has been described as an example, the specimen is not limited to the blood plasma separated from whole blood, and urine, saliva or the like can be used as the specimen as long as the detection subject substance is dispersed in the solution.

Meanwhile, in the respective embodiments, a case in which an antigen-antibody reaction is used has been described as an example, the combination of the detection subject substance and the substance that specifically bonds to the detection subject substance is not limited to an antigen and an antibody, and, for example, there are cases in which antibodies are detected using antigens, DNAs that carry out hybridization with specific DNAs are detected using the specific DNAs, DNA-bonded protein is bonded using DNAs, receptors are detected using ligands, lectin is detected using sugar, protease detection is used, a high-order structural change is used, or the like. Even in a case in which the combination of the detection subject substance and the substance that specifically bonds to the detection subject substance is not an antigen and an antibody, when two kinds of substances that specifically bond to different portions of the detection subject substance and metal nanoparticles are respectively bonded so as to configure the first complex and the second complex, and the first complex, the second complex and the detection subject substance are bonded so as to configure the third complex, the concentration of the detection subject substance can be measured using the bio-molecule detecting device according to the embodiment.

In addition, in the respective embodiments according to the invention, since measurement is possible in a liquid phase having the antigens 18, the antigens 40, the first complexes 20a, the second complexes 20b, the fourth complexes 20c and the fifth complexes 20d dispersed in a solution, compared with measurement in a solid phase, in which measurement is carried out with antibodies and the like fixed to a reaction layer, there is an advantage that the pretreatment is simple. In addition, since the antigens or complexes can freely move around in the solution, there is another advantage that the reaction is fast compared with measurement in a solid phase.

In addition, since the respective embodiments according to the invention do not investigate a change in the degree of polarization of fluorescence caused by a change of the Brownian motion unlike the fluorescence polarization method of the related art, and the components of a specimen only have a small influence on the measurement even when influencing the fluorescent service life of the fluorescent molecules.

Meanwhile, the respective embodiments according to the invention described above are to illustrate examples of the invention, and do not limit the configuration of the invention. The bio-molecule detecting device according to the invention is not limited to the respective embodiments, and can be modified in various manners and carried out within the scope of the object of the invention.

In addition, in the respective embodiments, the reagent holding unit in the reagent container 10 had a quadrangular prism-like shape, but the reagent holding unit does not necessarily need to have a quadrangular prism-like shape, and may have a cylindrical shape.

The bio-molecule detecting device and bio-molecule detecting method according to the invention can be used in, for example, a device that carries out the detection and quantity determination of a detection subject substance using an interaction between the detection subject substance and a substance that specifically bonds to the detection subject substance.

What is claimed is:

1. A bio-molecule detecting device comprising:
a container that holds a solution including first complexes having a first substance that can specifically bond to a specific portion on a bio-molecule, metal particles and a fluorescent molecule and second complexes having a second substance that can specifically bond to a portion different from the specific portion on the bio-molecule, metal particles and a fluorescent molecule and a specimen;
an orientation control unit that orients third complexes, in which the first complexes bond to the bio-molecules through the first substances and the second complexes bond to the bio-molecules through the second substances in at least two directions in the solution;
a light source that has a linear polarization component in a specific direction and radiates light, which causes surface plasmon resonance to the metal particles in the first complexes and the second complexes, on the solution;
a light-receiving unit that detects fluorescence emitted from the fluorescent molecules in the first complexes and the second complexes by an electric field generated by the surface plasmon resonance of the metal particles in the first complexes and the second complexes; and
a synchronous component-extracting unit that extracts a component synchronizing with an orientation cycle of the third complexes in the fluorescence detected by the light-receiving unit.

2. The bio-molecule detecting device according to claim 1,
wherein the orientation control unit includes an orientation control polarized light source that radiates linearly polarized light, which is different from the light radiated from the light source, on the solution; and
a polarizing axis-rotational moving unit that orients the third complexes in at least two directions in the solution by rotationally moving a polarizing axis of light radiated from the orientation control polarized light source.

3. The bio-molecule detecting device according to claim 2,
wherein the orientation control light source radiates linearly polarized light, which is different from the light radiated from the light source, on the solution from a plurality of locations.

4. The bio-molecule detecting device according to claim 1,
wherein the orientation control unit includes an orientation control light source that radiates light, which is different from the light radiated from the light source, on the solution; and
a switching unit that orients the third complexes in the solution in at least two directions by switching a radiation direction of light radiated from the orientation control light source.

5. The bio-molecule detecting device according to claim 4, comprising:
the orientation control light source that radiates light, which is different from the light radiated from the light source, on a plurality of locations of the solution.

6. The bio-molecule detecting device according to claim 1,
wherein the orientation control unit orients the third complexes in a first direction, in which a long-axis direction of the third complexes and a vibration direction of the light radiated from the light source become parallel, and
in a second direction, in which the long-axis direction of the third complexes and the vibration direction of the light radiated from the light source become vertical.

7. The bio-molecule detecting device according to claim 1,
wherein the orientation control unit changes the orientation direction of the third complexes at predetermined time intervals, and
the synchronous component-extracting unit extracts a component synchronizing with the orientation cycle of the third complexes by measuring the intensity of fluorescence generated from the solution including the third complexes a plurality of times.

8. The bio-molecule detecting device according to claim 7,
wherein the predetermined time interval is a time interval during which the orientation of all third complexes present in the solution is completed.

9. The bio-molecule detecting device according to claim 1,
wherein the wavelength of the light radiated from the light source is a wavelength that is not absorbed by the fluorescent molecule.

10. The bio-molecule detecting device according to claim 1,
wherein the synchronous component-extracting unit extracts a component synchronizing with the orientation cycle of the third complexes using the fact that the amount of fluorescence generated from the third complexes changes by the change of the orientation direction of the third complexes.

11. The bio-molecule detecting device according to claim 2,
wherein the solution is held in a container-holding unit having a flat plane at least in some part.

12. The bio-molecule detecting device according to claim 11,
wherein the orientation control polarized light source radiates linearly polarized light, which is different from the light radiated from the light source, in a direction in which the light outgoes from the flat plane of the container-holding unit through the solution, and focuses linearly polarized light, which is different from the light radiated from the light source, at an interface between the solution and the flat plane.

13. The bio-molecule detecting device according to claim 4,
wherein the solution is held in a container-holding unit having a flat plane at least in some part.

14. The bio-molecule detecting device according to claim 13,
wherein the orientation control light source radiates light, which is different from the light radiated from the light source, in a direction in which the light outgoes from the flat plane of the container-holding unit through the solution, and focuses light, which is different from the light radiated from the light source, at an interface between the solution and the flat plane.

15. A bio-molecule detecting method comprising:
by using the bio-molecule detecting device according to claim 1,
a step of mixing a solution including first complexes having a first substance that can specifically bond to a specific portion on a bio-molecule included in a specimen, metal particles and a fluorescent molecule and second complexes having a second substance that can specifically bond to a portion different from the specific portion on the bio-molecule, metal particles and a fluorescent molecule and a specimen;
a step of orienting third complexes, in which the first complexes bond to the bio-molecules through the first substances and the second complexes bond to the bio-molecules through the second substances in at least two directions in the solution;
a step of radiating light, which has a linearly polarized component in a specific direction and causes surface plasmon resonance to the metal particles in the first complexes and the second complexes, on the solution;
a step of detecting fluorescence emitted from the fluorescent molecules in the first complexes and the second complexes by an electric field generated by the surface plasmon resonance of the metal particles in the first complexes and the second complexes; and
a step of extracting a component synchronizing with an orientation cycle of the third complexes in the fluorescence detected by the light-receiving unit.
